SEQUENCE LISTING (3 sequences)

SEQ ID NO: 1 - DNA, 303 bp, Artificial Sequence
  Synthetic Probe
gaatgtgctgagttcagcaaatgtcttgacgctaagtcatcaccataactgcttagaaacccagaggaactcgggtgaagttaaataaataaggaccagccctctcgagcccctcccagagttattgcatttctcctctcaaggagcatggagtcccaatgggatgtgaagagcctcacctcccgtggcctttcagcagctgggagaggtcgtaatggggctctgaaggtagctcagcgcactgttcgtggagtggacagggatggagacggggaagttgaacacggtggtggtggaggtgct

SEQ ID NO: 2 - DNA, 20 bp, Artificial Sequence
  Synthetic oligonucleotide
ggaactcagtggaactcagg

SEQ ID NO: 3 - RNA, 14 nt, Artificial Sequence
  ribozyme sequence
nnnbngucnnnnnn

(19) United States
(12) Patent Application Publication
(10) Pub. No.: US 2003/0040505 A1
(43) Pub. Date: Feb. 27, 2003

(54) Synthetic phospholipids to ameliorate atherosclerosis and other inflammatory conditions

(75) Inventors: Alan M. Fogelman, Beverly Hills, CA (US); Mohamad Navab, Los Angeles, CA (US)

Correspondence Address: QUINE INTELLECTUAL PROPERTY LAW GROUP, P.C., P O BOX 458, ALAMEDA, CA, 94501, US

(73) Assignee: The Regents of the University of California

(21) Appl. No.: 09/994,227

(22) Filed: Nov. 26, 2001

Related U.S. Application Data
(63) Continuation-in-part of application No. 09/539,569, filed on Mar. 31, 2000.

(51) Int. Cl. A61K 31/685; A61K 31/66

(52) U.S. Cl. 514/78; 514/121

(57) ABSTRACT

This invention provides novel methods of inhibiting one or more symptoms of atherosclerosis. Also provided are assays for compounds that will inhibit the progression and/or ameliorate one or more symptoms of atherosclerosis. The methods and assays are based, in part, on the discovery that oxidized LDL or components thereof induce strong upregulation of MAP kinase phosphatase-1 which, in turn, is associated with an “inflammatory response” characteristic of atherosclerotic plaque formation. Inhibition of MKP-1 inhibits one or more symptoms of this response, e.g. monocyte adhesion, monocyte chemotaxis, differentiation into macrophages, etc. Inhibition of MKP-1 thus provides an effective method of inhibiting symptoms of atherosclerosis.

### CROSS-REFERENCE TO RELATED APPLICATIONS

This application is a continuation-in-part of U.S. Ser. No. 09/539,569, filed on Mar. 31, 2000, which is incorporated herein by reference in its entirety for all purposes.

### STATEMENT AS TO RIGHTS TO INVENTIONS MADE UNDER FEDERALLY SPONSORED RESEARCH AND DEVELOPMENT

This invention was made with Government support under Grant no: HL30568, awarded by the National Institutes of Health. The Government of the United States of America may have certain rights in this invention.

### FIELD OF THE INVENTION

This invention relates to the filed of atherosclerosis. In particular, this invention relates to the discovery that oxidized LDL upregulates MKP-1 resulting in an inflammatory response characteristic of atherosclerotic plaque formation.

### BACKGROUND OF THE INVENTION

Cardiovascular disease is a leading cause of morbidity and mortality, particularly in the United States and in Western European countries. Several causative factors are implicated in the development of cardiovascular disease including hereditary predisposition to the disease, gender, lifestyle factors such as smoking and diet, age, hypertension, and hyperlipidemia, including hypercholesterolemia. Several of these factors, particularly hyperlipidemia and hypercholesterolemia (high blood cholesterol concentrations) provide a significant risk factor associated with atherosclerosis.

Cholesterol is present in the blood as free and esterified cholesterol within lipoprotein particles, commonly known as chylomicrons, very low density lipoproteins (VLDL), low density lipoproteins (LDL), and high density lipoproteins (HDL). Concentration of total cholesterol in the blood is influenced by (1) absorption of cholesterol from the digestive tract, (2) synthesis of cholesterol from dietary constituents such as carbohydrates, proteins, fats and ethanol, and (3) removal of cholesterol from blood by tissues, especially the liver, and subsequent conversion of the cholesterol to bile acids, steroid hormones, and biliary cholesterol.

Maintenance of blood cholesterol concentrations is influenced by both genetic and environmental factors. Genetic factors include concentration of rate-limiting enzymes in cholesterol biosynthesis, concentration of receptors for low density lipoproteins in the liver, concentration of rate-limiting enzymes for conversion of cholesterols bile acids, rates of synthesis and secretion of lipoproteins and gender of person. Environmental factors influencing blood cholesterol concentration in humans include dietary composition, incidence of smoking, physical activity, and use of a variety of pharmaceutical agents. Dietary variables include amount and type of fat (saturated and polyunsaturated fatty acids), amount of cholesterol, amount and type of fiber, and perhaps amounts of vitamins such as vitamin C and D and minerals such as calcium.

As indicated above, high blood cholesterol concentration is one of the major risk factors for vascular disease and coronary heart disease in humans. Elevated low density lipoprotein cholesterol (“LDL-cholesterol”) and total cholesterol are directly related to an increased risk of coronary heart disease. Cholesterol and Mortality: 30 Years of Follow-Up from the Framingham Study, Anderson, Castelli, & Levy (1987) JAMA, 257: 2176-80.

Although high levels of total cholesterol and LDL-cholesterol are risk factors in developing atherosclerosis and vascular diseases, a deficiency of high density lipoprotein cholesterol (hereafter “HDL-cholesterol”) has recently been recognized as a risk factor for developing these conditions. Several clinical trials support a protective role of HDL-cholesterol against atherosclerosis. A study has shown that for every 1-mg/dl increase in HDL-cholesterol in the blood, the risk for coronary vascular disease is decreased by 3% in women. High-density Lipoprotein Cholesterol and Cardiovascular Disease: Four Prospective American Studies, Gordon, Probstfield, and Garrison et al. (1989) Circulation, 79: 8-15.

It is widely believed that HDL is a “protective” lipoprotein (Vega and Grundy (1996) Curr. Opin. Lipidology, 7: 209-216) and that increasing plasma levels of HDL may offer a direct protection against the development of atherosclerosis. Numerous studies have demonstrated that both the risk of coronary heart disease (CHD) in humans and the severity of experimental atherosclerosis in animals are inversely correlated with serum HDL cholesterol (HDL-C) concentrations (Russ et al. (1951) Am. J. Med., 11: 480-493; Gofman et al. (1966) Circulation, 34: 679-697; Miller and Miller (1975) Lancet, 1: 16-19; Gordon et al. (1989) Circulation, 79: 8-15; Stampfer et al. (1991) N. Engl. J. Med., 325: 373-381; Badimon et al. (1989) Lab. Invest., 60: 455-461).

While HDL/LDL ratios have appear to provide a good marker for risk of atherosclerosis and heart disease on a population level, HDL and/or LDL measurements have proven to be poor prognostic indicators at an individual level. In particular individuals with high HDL:LDL ratios have been observed with severe atherosclerosis, while conversely, individuals with very low HDL:LDL ratios have been identified with no-evidence of atherosclerosis.

### SUMMARY OF THE INVENTION

This invention provides novel methods of inhibiting one or more symptoms of atherosclerosis. Also provided are assays for compounds that will inhibit the progression and/or ameliorate one or more symptoms of atherosclerosis. The methods and assays are based, in part, on the discovery that oxidized LDL or components thereof induce strong upregulation of MAP kinase phosphatase-1 which, in turn, is associated with an “inflammatory response” characteristic of atherosclerotic plaque formation. Inhibition of MKP-1 inhibits one or more symptoms of this response, e.g. monocyte adhesion, monocyte chemotaxis, differentiation into macrophages, etc. Inhibition of MKP-1 thus provides an effective method of inhibiting symptoms of atherosclerosis.

Thus, in one embodiment, this invention provides methods of identifying a compound that ameliorates one or more symptoms of atherosclerosis (or other inflammatory diseases, e.g. rheumatoid arthritis, idiopathic pulmonary fibrosis, lupus, and the like). The methods involve contacting a cell comprising an MKP-1 gene with a test agent; and detecting expression of the MKP-1 gene whereby an inhibition of expression of MKP-1 indicates that the test agent is a compound that ameliorates one or more symptoms of atherosclerosis. In certain embodiments, the methods further involve contacting the cell with an oxidized low-density lipoprotein (Ox-LDL) or a component thereof including an oxidized phospholipid that upregulates expression of a MAP kinase phosphatase 1 (MKP-1). In these embodiments, detecting preferably comprises detecting expression of the MKP-1 gene whereby an inhibition of expression in the cell contacted with the test agent as compared to the cell contacted with the oxidized low density lipoprotein (Ox-LDL) or component thereof and no test agent (or the test agent at a reduced concentration) indicates that the test agent is a compound that ameliorates one or more symptoms of atherosclerosis. It will be appreciated that in certain embodiments, multiple test agents can be assayed simultaneously with the same cell. Where a positive result is obtained for the plurality of test agents, each agent comprising the plurality can be retested individually.

In particularly preferred assays, the low density lipoprotein (Ox-LDL) or a component thereof, is an oxidized phospholipid that is an oxidized form of one or more lipids selected from the group consisting of oxidized 1-palmitoyl-2-arachidonoyl-sn-glycero-3-phosphorylcholine (Ox-PAPC), 1-palmitoyl-2-oxovaleroyl-sn-glycero-3-phosphorylcholine (POVPC), 1-palmitoyl-2-glutaroyl-sn-glycero-3-phosphorylcholine (PGPC), 1-palmitoyl-2-epoxyisoprostane-sn-glycero-3-phosphorylcholine (PEIPC), 1-stearoyl-2-arachidonoyl-sn-glycero-3-phosphorylcholine (Ox-SAPC), 1-stearoyl-2-oxovaleroyl-sn-glycero-3-phosphorylcholine (SOVPC), 1-stearoyl-2-glutaroyl-sn-glycero-3-phosphorylcholine (SGPC), 1-stearoyl-2-epoxyisoprostane-sn-glycero-3-phosphorylcholine (SEIPC), 1-stearoyl-2-arachidonyl-sn-glycero-3-phosphorylethanolamine (Ox-SAPE), 1-stearoyl-2-oxovaleroyl-sn-glycero-3-phosphorylethanolamine (SOVPE), 1-stearoyl-2-glutaroyl-sn-glycero-3-phosphorylethanolamine (SGPE), and 1-stearoyl-2-epoxyisoprostane-sn-glycero-3-phosphorylethanolamine(SEI PE).

In preferred embodiments, the cell is a mammalian cell, more preferably a human cell. Particularly preferred cells include cells of a human blood vessel. Particularly preferred cells are cells cultured ex vivo.

Detecting MKP-1 expression preferably involves detecting an MKP-1 nucleic acid (e.g. MKP-1 mRNA, cDNA, cRNA, etc.) or fragments thereof, detecting an MKP-1 polypeptide or fragments thereof, or measuring MKP-1 polypeptide activity. In a particularly preferred embodiment, detecting MKP-1 protein activity comprises detecting an atherosclerotic symptom (e.g. monocyte binding, monocyte chemotaxis, etc.). In a particularly preferred embodiment the expression level of MKP-1 is detected by measuring the level of MKP-1 mRNA in said cell. In particularly preferred embodiments, the level of MKP-1 mRNA is measured by hybridizing said mRNA to a probe that specifically hybridizes to an MKP-1 nucleic acid. Preferred hybridization formats include, but are not limited to a Northern blot, a Southern blot using DNA derived from the MKP-1 RNA, an array hybridization, an affinity chromatography, and an in situ hybridization. The hybridization may be in an array based format in which case the probe that specifically binds to an MKP-1 nucleic acid is one or more members of a plurality of probes that forms an array of probes. The level of MKP-1 mRNA is measured in some embodiments using a nucleic acid amplification reaction (e.g. PCR). In other embodiments, the level of MKP-1 expression is determined by detecting the expression level of a MKP-1 protein in the biological sample. Preferred protein detection methods include, but are not limited to capillary electrophoresis, a Western blot, mass spectroscopy, ELISA, immunochromatography, and immunohistochemistry.

In certain embodiments, the test agent is contacted to cells in culture, while in other embodiments, the test agent is administered to an animal comprising a cell containing the MKP-1 nucleic acid or the MKP-1 protein. While essentially any molecule composition or combination molecules may comprise a test agent, preferred test agents are not antibody and/or not proteins. Particularly preferred test agents are small organic molecules.

In preferred embodiments, the methods further involve recording test agents that alter expression of the MKP-1 nucleic acid or the MKP-1 protein in a database of modulators of MKP-1 activity or in a database of agents that ameliorate one or more symptoms of atherosclerosis.

In another embodiment, this invention provides methods of prescreening for a modulator or inhibitor of a MKP-1. These methods involve contacting an MKP-1 nucleic acid or an MKP-1 protein with a test agent; and detecting specific binding of the test agent to the MKP-1 protein or nucleic acid where specific binding of the test agent to the MKP-1 protein or nucleic acid indicates that said test agent is a potential modulator of MKP-1. In certain embodiments, the method further involves recording test agents that specifically bind to the MKP-1 nucleic acid or to the MKP-1 protein in a database of candidate modulators of MKP-1 activity, and/or in a database of agents that ameliorate one or more symptoms of atherosclerosis. In particularly preferred embodiments, the test agent is not an antibody and/or not a protein. Particularly preferred test agents include hydrophobic compounds, and/or lipids, and/or small organic molecules. Binding detection is by any of a number of methods known to those of skill in the art. Thus, for example, in some embodiments the detecting comprises detecting specific binding of the test agent to the MKP-1 nucleic acid (e.g. using a method selected from the group consisting of a Northern blot, a Southern blot using DNA, an array hybridization, an affinity chromatography, and an in situ hybridization). In other embodiments, the detecting comprises detecting specific binding of the test agent to said MKP-1 protein (e.g., via a method selected from the group consisting of capillary electrophoresis, a Western blot, mass spectroscopy, ELISA, immunochromatography, and immunohistochemistry).

Depending on the assay format, the test agent is contacted directly to the MKP-1 nucleic acid or to the MKP-1 protein, or the test agent is contacted to a cell containing the MKP-1 nucleic acid (e.g. ex vivo, in culture) or the MKP-1 protein, or the test agent is contacted to (e.g., administered to) an animal comprising a cell containing the MKP-1 nucleic acid or the MKP-1 protein.

In another embodiment, this invention provides methods of ameliorating one or more symptoms of atherosclerosis. The methods involve inhibiting expression of the MAP kinase phosphatase 1 (MKP-1). The MKP-1 expression can be inhibited in all contexts, or inhibition can be tissue specific, or at specific times, or in response to specific stimuli. In one embodiment, the inhibition of MKP-1 comprises directly or indirectly inhibiting the upregulation of MKP-1 that typically occurs in response to an oxidized low density lipoprotein (Ox-LDL) or a component thereof (e.g. including an oxidized phospholipid).

The MKP-1 inhibition can be by any of a variety of methods including, but not limited to contacting an MKP-1 nucleic acid with an antisense oligonucleotide, contacting an MKP-1 nucleic acid with a ribozyme and/or catalytic DNA, transfecting a cell comprising an MKP-1 gene with a nucleic acid that inactivates the MKP-1 gene by homologous recombination with the MKP-1 gene, the MKP-1 promoter, or intervening nucleic acids, transfecting a cell comprising an MKP-1 gene with a nucleic acid encoding an antibody that specifically binds an MKP-1 polypeptide, contacting a cell comprising an MKP-1 gene with a small organic molecule that inhibits upregulation of the MKP-1 gene (e.g. upregulation that typically occurs in response to an oxidized LDL or a component thereof comprising an oxidized phospholipid), and contacting a cell comprising an MKP-1 gene with a phospholipid that, directly or indirectly, inhibits upregulation of the MKP-1 gene.

In certain embodiments, the methods are practiced in non-human mammals, but in particularly preferred embodiments, the methods are practiced in humans. Preferred humans are human patients (subjects) diagnosed as having, or at risk for, atherosclerosis. Particularly preferred humans include human patients diagnosed as having atherosclerosis. In certain embodiments the methods are practiced in human subjects not diagnosed as having a cancer or at risk for a cancer or other neoplasm and/or subjects diagnosed as in remission or “cured” of a cancer.

This invention also provides kits for practicing the assay and “treatment” methods of this invention. Thus, in one embodiment this invention provides kits for screening for compounds that ameliorate one or more symptoms of atherosclerosis (e.g. monocyte adhesion, monocyte chemotaxis, monocyte differentiation into macrophages, etc.). The kits preferably include a cell that comprises an MKP-1 nucleic acid; and a detection moiety selected from the group consisting of a labeled antibody that specifically binds to an MKP-1 polypeptide, a nucleic acid that specifically binds to an MKP-1 nucleic acid, and a primer that specifically amplifies an MKP-1 nucleic acid or a fragment thereof. In some embodiments, the kits further comprise an oxidized low density lipoprotein or a component thereof comprising an oxidized phospholipid. Preferred oxidized phospholipids include, but are not limited to those described herein. The kit, optionally, further includes instructional materials providing protocols for screening for inhibitors of MKP-1 and, optionally, teaching that such inhibitors ameliorate one or more symptoms of atherosclerosis and/or associated pathologies, and/or rheumatoid arthritis, and/or other inflammatory processes.

In another embodiment, this invention provides a method of ameliorating one or more symptoms of atherosclerosis in a mammal (e.g. human, non-human primate, horse, cow, cat, dog, etc.). The method involves administering to the mammal one or more phospholipids in an amount sufficient to ameliorate one or more symptoms of atherosclerosis. In certain embodiments, preferred phospholipids are phospholipids that inhibit upregulation of an MKP-1 gene. In certain embodiments, the phospholipid is a phospholipid selected from the group consisting of phosphatidyl choline, phosphatidyl serine, phosphatidyl ethanolamine, and phosphatidyl inositol. Particularly preferred phospholipids are these phospholipids where the phospholipids comprise independently selected fatty acids in the sn-1 and sn-2 positions ranging in length from about 4 to about 24 carbons. Preferred fatty acids include, but are not limited to propionoyl, butanoyl, pentanoyl, caproyl, heptanoyl, capryloyl, nonanoyl, capryl, undcanoyl, lauroyl, tridecanoyl, and myristoyl. One particularly preferred phospholipid is 1,2-dimyristoyl-sn-glycero-3-phosphocholine or an analogue or derivative thereof. The phospholipid(s) can be provided in a unit dose form. In certain embodiments, the phospholipid is provided in a combination of phospholipids. The administration can be by a route selected from the group consisting of oral administration, nasal administration, rectal administration, intraperitoneal injection, and intravascular injection, subcutaneous injection, transcutaneous administration, and intramuscular injection. Particularly preferred methods utilize oral administration or an injection. In certain embodiments, the symptoms of atherosclerosis are in a human patient diagnosed as having or as at risk for atherosclerosis. In certain embodiments, the mammal can also be administered a statin. In certain embodiments, the mammal is not administered a D peptide and/or a synthetic peptide. In certain embodiments, the mammal is not simultaneously administered a D peptide and/or a synthetic peptide. In certain embodiments, the phospholipid is administered in a formulation lacking other active agents.

This invention also provides a method of mitigating or preventing a coronary complication (e.g. a symptom of atherosclerosis) associated with an acute phase response to an inflammation in a mammal. The method involves administering (e.g. in a unit dose form) to a mammal having the acute phase response, or at risk for the acute phase response, one or more phospholipids in an amount sufficient to mitigate or prevent said coronary complication. Preferred phospholipids include, but are not limited to those described above. Preferred routes of administration include, but are not limited to oral administration, nasal administration, rectal administration, intraperitoneal injection, and intravascular injection, subcutaneous injection, transcutaneous administration, and intramuscular injection. The inflammatory response can be an inflammatory response associated with a recurrent inflammatory disease (e.g. leprosy, tuberculosis, systemic lupus erythematosus, polymyalgia rheumatica, polyarteritis nodosa, scleroderma, idiopathic pulmonary fibrosis, chronic obstructive pulmonary disease, Alzheimers Disease AIDS, coronary calcification, calcific aortic stenosis, osteoporosis, rheumatoid arthritis, etc.). The acute phase response can be an inflammatory response associated with a condition selected from the group consisting of a bacterial infection, a viral infection, a fungal infection, an organ transplant, a wound, an implanted prosthesis, parasitic infection, sepsis, endotoxic shock syndrome, and biofilm formation.

This invention also provides a method of mitigating or preventing a coronary complication associated with an acute phase response to an inflammation in a mammal, where said coronary complication is a symptom of atherosclerosis, where the method involves assaying the mammal for an acute phase protein (APP) level indicative of an acute phase response or a significant risk of an acute phase response; and administering to a mammal showing an acute phase protein (APP) level indicative of an acute phase response one or more phospholipids in an amount sufficient to mitigate or prevent said coronary complication. Preferred phospholipids and/or routes of administration include, but are not limited to those described above. In certain embodiments, the acute phase response is an inflammatory response is a response associated with a recurrent inflammatory disease, a bacterial infection, a viral infection, a fungal infection, an organ transplant, a wound, an implanted prosthesis, parasitic infection, sepsis, endotoxic shock syndrome, and/or biofilm formation. The acute phase protein (APP) can be a positive APR (e.g., serum amyloid A, c-reactive protein, serum amyloid P component, C2 complement protein, C3 complement protein, C4 complement protein, C5 complement protein, C9 complement protein, B complement protein, C1 inhibitor, C4 binding protein, fibrinogen, von Willebrand factor, a1-antitrypsin, a1-antichymotrypsin, a2 antiplasmin, heparin cofactor II, plasminogen activator inhibitor I, haptoglobin, haemopexin, ceruloplasmin, manganese superoxide dismutase, a1-acid glycoprotein, haeme oxygenase, mannose binding protein, leukocyte protein I, lipoprotein (a), lipopolysaccharide binding protein, etc.). The acute phase protein (APP) can be a negative APR (e.g. albumin, prealbumin, transferin, apoAI, apoAII, a2-HS glycoprotein, inter-a-trypsin inhibitor, histidine rich glycoprotein, etc.).

In still yet another embodiment, this invention provides a method of inhibiting a symptom of an inflammatory condition. The method involves administering to a mammal exhibiting a symptom of a pathology characterized by an inflammatory response one or more phospholipid(s) in an amount sufficient to mitigate a symptom associated with the inflammatory condition. Preferred phospholipids and/or routes of administration include, but are not limited to those described above. In certain embodiments, the symptoms are in a human patient diagnosed as having or at risk for atherosclerosis. In certain embodiments, the inflammatory condition rheumatoid arthritis, lupus erythematous, polyarteritis nodosa, osteoporosis, and/or a viral illness.

This invention also provides a composition for ameliorating one or more symptoms of atherosclerosis. The composition preferably comprises one or more phospholipid(s) in a unit dose form. The unit dose form can further comprise a pharmacologically acceptable excipient. Preferred phospholipids include, but are not limited to one or more of the phospholipids described above (e.g. 1,2-dimyristoyl-sn-glycero-3-phosphocholine or an analogue thereof).

This invention also provides a kit for ameliorating one or more symptoms of atherosclerosis. Preferred kits include one or more phospholipids; and instructional materials teaching the administration of said phospholipid to a mammal to mitigate one or more symptoms of atherosclerosis. Preferred phospholipids include, but are not limited to one or more of the phospholipids described above (e.g. 1,2-dimyristoyl-sn-glycero-3-phosphocholine or an analogue thereof). The phospholipid(s) can be provided in a unit dose form.

In still another embodiment, this invention provides a kit for inhibiting a symptom of an inflammatory condition. Preferred kits include, but are not limited to a phospholipid; and

instructional materials teaching the administration of the phospholipid to a mammal to mitigate one or more symptoms of an inflammatory condition. Preferred phospholipids include, but are not limited to one or more of the phospholipids described above (e.g. 1,2-dimyristoyl-sn-glycero-3-phosphocholine or an analogue thereof). The phospholipid(s) can be provided in a unit dose form.

In another embodiment, this invention provides a method of repairing tissue damage (e.g. lesions) associated with atherosclerosis. The method involves administering to a mammal suffering such tissue damage a phospholipid in an amount sufficient to partially or fully repair said tissue damage. Preferred phospholipids include, but are not limited to one or more of the phospholipids described above (e.g. 1,2-dimyristoyl-sn-glycero-3-phosphocholine or an analogue thereof). The phospholipid(s) can be provided in a unit dose form. Preferred routes of administration include, but are not limited to oral administration, nasal administration, rectal administration, intraperitoneal injection, and intravascular injection, subcutaneous injection, transcutaneous administration, and intramuscular injection. In certain embodiments, the symptoms are in a human patient diagnosed as having or at risk for atherosclerosis.

In another embodiment this invention provides a kit for inhibiting expression of MKP-1. Preferred kits comprise an inhibitor of MKP-1 selected from the group consisting of an MKP-1 antisense molecule, an MKP-1 ribozyme, a lipid that inhibits upregulation of MKP-1 in response to oxidized phospholipids, an antibody that binds to and blocks MKP-1 activity. Such kits, optionally, further comprise instructional materials teaching inhibition of MKP-1 as a method of ameliorating one or more symptoms of atherosclerosis and/or rheumatoid arthritis, and/or other inflammatory processes.

Definitions.

“Atherosclerosis” is the process of accumulation of cholesterol, and/or other lipids, and/or macrophages, and/or smooth muscle cells within/on the arterial wall which results in the occlusion, or stenosis, of coronary and cerebral arterial vessels often by plaque erosion and/or rupture with a resulting thrombus that often leads to subsequent myocardial infarction and stroke.

The terms “polypeptide”, “peptide” and “protein” are used interchangeably herein to refer to a polymer of amino acid residues. The terms apply to amino acid polymers in which one or more amino acid residue is an artificial chemical analogue of a corresponding naturally occurring amino acid, as well as to naturally occurring amino acid polymers.

The term “MKP-1 nucleic acid” refers to a nucleic acid encoding a MAP kinase phosphatase 1 (MKP-1) (see, e.g., GenBank Accession No: x68277) or to a nucleic acid derived therefrom. Thus, MKP-1 nucleic acids include, but are not limited, to the MKP-1 gene (e.g. erp/mkp-1), an MKP-1 RNA, an MKP-1 cDNA, an MKP-1 cRNA, and the like.

An “MKP-1 protein or polypeptide” is a protein expressed by an MKP-1 gene or cDNA, e.g. a MAP kinase phosphatase 1.

The term “inhibit expression” is used with reference to inhibition of MKP-1 to refer to a reduction or blocking of MKP-1 transcription, and/or translation, and/or formation or availability of active MKP-1 protein.

The term an MKP-1 nucleic acid refers to a nucleic acid encoding MKP-1 or a fragment thereof or to a nucleic acid complementary to a nucleic acid encoding MKP-1 or a fragment thereof. MKP-1 nucleic acids include, but are not limited to MKP-1 genomic DNA, mRNA, cDNA, cRNA, or fragments thereof.

The term “detecting an MKP-1 mRNA or cDNA” refers to detecting and/or quantifying a MKP-1 nucleic acid or a nucleic acid derived therefrom the quantification of which provides an indication of the expression level of the MKP-1 nucleic acid. The term thus includes, but is not limited to detection of MKP-1 mRNA, cDNA, MKP-1 amplification products, and fragments of any of these.

The terms “binding partner”, or “capture agent”, or a member of a “binding pair” refers to molecules that specifically bind other molecules to form a binding complex such as antibody-antigen, lectin-carbohydrate, nucleic acid-nucleic acid, biotin-avidin, etc.

The term “specifically binds”, as used herein, when referring to a biomolecule (e.g., protein, nucleic acid, antibody, etc.), refers to a binding reaction which is determinative of the presence biomolecule in heterogeneous population of molecules (e.g., proteins and other biologics). Thus, under designated conditions (e.g. immunoassay conditions in the case of an antibody or stringent hybridization conditions in the case of a nucleic acid), the specified ligand or antibody binds to its particular “target” molecule and does not bind in a significant amount to other molecules present in the sample.

The phrase “symptom of atherosclerosis” refers to one or more symptoms characteristic of atherosclerotic plaque formation and associated pathologies. Such symptoms include, but are not limited to monocyte binding to the vascular wall, monocyte chemotaxis into the subendothelial space, monocyte differentiation into macrophages, vascular occlusion, elevated blood pressure associated with vascular occlusion, stiffening of the vascular wall, stroke, and the like causing inflammation and in some instances plaque rupture or plaque erosion with subsequent thrombosis”.

The terms “nucleic acid” or “oligonucleotide” or grammatical equivalents herein refer to at least two nucleotides covalently linked together. A nucleic acid of the present invention is preferably single-stranded or double stranded and will generally contain phosphodiester bonds, although in some cases, as outlined below, nucleic acid analogs are included that may have alternate backbones, comprising, for example, phosphoramide (Beaucage et al. (1993) Tetrahedron 49(10): 1925) and references therein; Letsinger (1970) J. Org. Chem. 35:3800; Sprinzl et al. (1977) Eur. J. Biochem. 81: 579; Letsinger et al. (1986) Nucl. Acids Res. 14: 3487; Sawai et al. (1984) Chem. Lett. 805, Letsinger et al. (1988) J. Am. Chem. Soc. 110: 4470; and Pauwels et al. (1986) Chemica Scripta 26: 141 9), phosphorothioate (Mag et al. (1991) Nucleic Acids Res. 19:1437; and U.S. Pat. No. 5,644,048), phosphorodithioate (Briu et al. (1989) J. Am. Chem. Soc. 111 :2321, 0-methylphophoroamidite linkages (see Eckstein, Oligonucleotides and Analogues: A Practical Approach, Oxford University Press), and peptide nucleic acid backbones and linkages (see Egholm (1992) J. Am. Chem. Soc. 114:1895; Meier et al. (1992) Chem. Int. Ed. Engl. 31: 1008; Nielsen (1993) Nature, 365: 566; Carlsson et al. (1996) Nature 380: 207). Other analog nucleic acids include those with positive backbones (Denpcy et al. (1995) Proc. Natl. Acad. Sci. USA 92: 6097; non-ionic backbones (U.S. Pat. Nos. 5,386,023, 5,637,684, 5,602,240, 5,216,141 and 4,469,863; Angew. (1991) Chem. Intl. Ed. English 30: 423; Letsinger et al. (1988) J. Am. Chem. Soc. 110: 4470; Letsinger et al. (1994) Nucleoside & Nucleotide 13:1597; Chapters 2 and 3, ASC Symposium Series 580, “Carbohydrate Modifications in Antisense Research”, Ed. Y. S. Sanghui and P. Dan Cook; Mesmaeker et al. (1994), Bioorganic & Medicinal Chem. Lett. 4: 395; Jeffs et al. (1994) J. Biomolecular NMR 34:17; Tetrahedron Lett. 37:743 (1996)) and non-ribose backbones, including those described in U.S. Pat. Nos. 5,235,033 and 5,034,506, and Chapters 6 and 7, ASC Symposium Series 580, Carbohydrate Modifications in Antisense Research, Ed. Y. S. Sanghui and P. Dan Cook. Nucleic acids containing one or more carbocyclic sugars are also included within the definition of nucleic acids (see Jenkins et al. (1995), Chem. Soc. Rev. pp169-176). Several nucleic acid analogs are described in Rawls, C & E News Jun. 2, 1997 page 35. These modifications of the ribose-phosphate backbone may be done to facilitate the addition of additional moieties such as labels, or to increase the stability and half-life of such molecules in physiological environments.

The terms “hybridizing specifically to” and “specific hybridization” and “selectively hybridize to,” as used herein refer to the binding, duplexing, or hybridizing of a nucleic acid molecule preferentially to a particular nucleotide sequence under stringent conditions. The term “stringent conditions” refers to conditions under which a probe will hybridize preferentially to its target subsequence, and to a lesser extent to, or not at all to, other sequences. Stringent hybridization and stringent hybridization wash conditions in the context of nucleic acid hybridization are sequence dependent, and are different under different environmental parameters. An extensive guide to the hybridization of nucleic acids is found in, e.g., Tijssen (1993) Laboratory Techniques in Biochemistry and Molecular Biology—Hybridization with Nucleic Acid Probes part I, chapt 2, Overview of principles of hybridization and the strategy of nucleic acid probe assays, Elsevier, N.Y. ( Tijssen). Generally, highly stringent hybridization and wash conditions are selected to be about 5° C. lower than the thermal melting point (Tₘ) for the specific sequence at a defined ionic strength and pH. The Tₘ is the temperature (under defined ionic strength and pH) at which 50% of the target sequence hybridizes to a perfectly matched probe. Very stringent conditions are selected to be equal to the Tₘ for a particular probe. An example of stringent hybridization conditions for hybridization of complementary nucleic acids which have more than 100 complementary residues on an array or on a filter in a Southern or northern blot is 42° C. using standard hybridization solutions (see, e.g., Sambrook (1989) Molecular Cloning: A Laboratory Manual (2 nd ed.) Vol. 1-3, Cold Spring Harbor Laboratory, Cold Spring Harbor Press, NY, and detailed discussion, below), with the hybridization being carried out overnight. An example of highly stringent wash conditions is 0.15 M NaCl at 72° C. for about 15 minutes. An example of stringent wash conditions is a 0.2× SSC wash at 65° C. for 15 minutes (see, e.g., Sambrook supra.) for a description of SSC buffer). Often, a high stringency wash is preceded by a low stringency wash to remove background probe signal. An example medium stringency wash for a duplex of, e.g., more than 100 nucleotides, is 1× SSC at 45° C. for 15 minutes. An example of a low stringency wash for a duplex of, e.g., more than 100 nucleotides, is 4× to 6× SSC at 40° C. for 15 minutes.

The term “test agent” refers to an agent that is to be screened in one or more of the assays described herein. The agent can be virtually any chemical compound. It can exist as a single isolated compound or can be a member of a chemical (e.g. combinatorial) library. In a particularly preferred embodiment, the test agent will be a small organic molecule.

The term “small organic molecule” refers to a molecule of a size comparable to those organic molecules generally used in pharmaceuticals. The term excludes biological macromolecules (e.g., proteins, nucleic acids, etc.). Preferred small organic molecules range in size up to about 5000 Da, more preferably up to 2000 Da, and most preferably up to about 1000 Da.

The term database refers to a means for recording and retrieving information. In preferred embodiments the database also provides means for sorting and/or searching the stored information. The database can comprise any convenient media including, but not limited to, paper systems, card systems, mechanical systems, electronic systems, optical systems, magnetic systems or combinations thereof. Preferred databases include electronic (e.g. computer-based) databases. Computer systems for use in storage and manipulation of databases are well known to those of skill in the art and include, but are not limited to “personal computer systems”, mainframe systems, distributed nodes on an inter- or intra-net, data or databases stored in specialized hardware (e.g. in microchips), and the like.

### BRIEF DESCRIPTION OF THE DRAWINGS

FIG. 1 illustrates the dose dependent induction of MKP-1 as well as Gro-α, IL-8, and Annexin II in response to oxidized L-α-1-palmitoyl-2-arachidonoyl-sn-glycero-3-phosphocholine (PAPC) presented to human aortic endothelial cells in culture.

FIG. 2 demonstrates the induction of MKP1 by oxidized PAPC (Ox-PAPC) in human aortic endothelial cells as a function of time.

FIG. 3A and FIG. 3B (two separate experiments) demonstrate by Western blotting that antisense oligonucleotides to MKP-1 (but not sense oligonucleotides) prevent Ox-PAPC-induced MKP-1 protein expression in human aortic endothelial cells.

FIG. 4 demonstrates, by Western blotting, that antisense oligonucleotides to MKP-1 (but not sense oligonucleotides) prevent the Ox-PAPC induction of monocyte adherence to human aortic endothelial cells.

FIG. 5A, FIG. 5B, and FIG. 5C (three different experiments) demonstrate that antisense oligonucleotides to MKP-1 (but not sense oligonucleotides) prevent the secretion of monocyte chemotactic activity by human aortic endothelial cells exposed to Ox-PAPC in culture.

FIG. 6 illustrates the effect of 1,2-dimyristoyl-sn-glycero-3-phosphocholine (1,2-ditetradecanoyl-sn-glycero-3-phosphocholine) and egg yolk lecithin on lipoprotein cholesterol levels in apo E deficient mice. The abbreviation CCP represents cholesterol containing particles in a size range that is smaller than mature HDL. Since these particles were only seen after 24 h and not at 48 h, it is presumed that they represent pre-HDL particles.

FIG. 7 illustrates the effect of 1,2-dimyristoyl-sn-glycero-3-phosphocholine (1,2-ditetradecanoyl-sn-glycero-3-phosphocholine) (shown in the figure as squares and abbreviated as DGP) and egg yolk lecithin (shown in the figure as asterisks) on paraoxonase activity. The control samples are shown as diamonds in the figure.

FIG. 8 illustrates the effect of 1,2-dimyristoyl-sn-glycero-3-phosphocholine (1,2-ditetradecanoyl-sn-glycero-3-phosphocholine) on HDL protective capacity and LDL susceptibility to oxidation. The values are mean ±SD of the number of monocytes counted in 9 high power fields from quadruple wells. Asterisks indicate significant differences as compared with controls at a level of p<0.001.

FIG. 9 illustrates the effect of 1,2-dimyristoyl-sn-glycero-3-phosphocholine (1,2-ditetradecanoyl-sn-glycero-3-phosphocholine) on fatty streak lesions.

FIG. 10 Oral DGP causes lesion regression in mice.

### DETAILED DESCRIPTION

This invention provides novel methods of inhibiting one or more symptoms of atherosclerosis. Also provided are assays for compounds that will inhibit the progression and/or ameliorate one or more symptoms of atherosclerosis. The methods and assays are based, in part, on elucidation of a mechanism by which oxidized lipids (e.g. lipids and other fractions of mildly or highly oxidized LDL) induce the inflammatory response characteristic of atherosclerotic plaque formation.

It has been noted that freshly isolated low density lipoprotein (LDL) contains lipid hydroperoxides (Sevanian et al. (1997) J. Lipid Res., 38: 419-428). We believe that LDL oxidation in vivo requires that the LDL be “seeded” with reactive species before it can be oxidized. The presence of oxidized lipids results in an “inflammatory response; the induction of monocyte binding, chemotaxis, and differentiation into macrophages. This process underlies plaque formation characteristic of atherosclerosis and other inflammatory diseases.

More particularly, without being bound to a particular theory, it is believed that the biologically active lipids in mildly oxidized LDL (m/z 594, 610, and 828) are formed in a series of three steps. The first step is the seeding of LDL with products of the metabolism of linoleic and arachidonic acid as well as with cholesteryl hydroperoxides. The second step involves trapping of LDL in the subendothelial space and the delivery to this trapped LDL of additional reactive oxygen species derived from nearby artery wall cells. The third step is the non-enzymatic oxidation of LDL phospholipids that occurs when a critical threshold of “seeding molecules” (e.g. 13-hydroperoxyoctadecadienoic acid [13(S)-HPODE] and 15-hydroperoxyeicosatetrenoic acid [15(S)-HPETE]) is reached in the LDL. This results in the formation of specific oxidized lipids (m/z 594,610, 828) that induce monocyte binding, chemotaxis, and differentiation into macrophages.

It was a discovery of this invention that the “inflammatory response” (e.g. induction of monocyte binding, chemotaxis, and differentiation into macrophages is mediated by strong upregulation of the regulated phosphatase (ERP) also known as MAP kinase phosphatase 1 (MKP-1). In particular it is demonstrated herein, that mildly oxidized or highly oxidized low density lipoproteins (LDLs) or components thereof, in particular oxidized phospholipids induce a strong upregulation of the MKP-1 gene. Upregulation of this gene results in an “inflammatory response” characteristic of plaque formation and/or inflammation associated with rheumatoid arthritis, or other inflammatory conditions. In particular, upregulation of the MKP-gene results in monocyte adhesion, chemotaxis and differentiation into macrophages in a manner essentially identical to that observed in atherosclerotic plaque formation.

Moreover, it is demonstrated herein that inhibition of MKP-1 blocks this inflammatory response and effectively protects the vascular endothelium from adverse effects of oxidized lipids, and/or mildly or highly oxidized LDL. It has been demonstrated (e.g. by the production of MKP-1) knockout mice that inhibition of MKP-1 appears to have no serious adverse effects on the organism. (Indeed, because of this lack of “detectable effects” the function of MKP-1 in an organism was unknown). Because of the apparent lack of adverse effects associated with knocking out or inhibiting MKP-1, blocking or downregulation of this gene provides and effective method of ameliorating one or more symptoms of atherosclerosis and/or rheumatoid arthritis.

Thus, in one embodiment, this invention provides methods of ameliorating one or more symptoms of atherosclerosis and/or rheumatoid arthritis. The methods involve blocking, knocking out, or inhibiting MKP-1 expression. In particularly preferred embodiments, the blocking of MKP-1 is specific blocking of the response of MKP-1 to oxidized LDL and/or components thereof. The blocking or inhibition can be at the level of transcription, translation, or the active protein can be inhibited/antagonized.

In view of the teaching provided herein MKP-1 is a good target for screening for agents useful in the treatment of atherosclerosis, rheumatoid arthritis, or other inflammatory processes/pathologies. Typically, the methods involve contacting a nucleic acid, and/or a cell, and/or a tissue, and/or an organ, and/or an organism (e.g. mammal) with one or more test agents and evaluating the ability of those agent(s) to block, more preferably to specifically block, MKP-1 transcription, MKP-1 translation, or activity of the MKP-1 protein. Because it has been demonstrated herein, that MKP-1 is strongly upregulated in the presence of oxidized phospholipids, it is convenient to run assays for the MKP-1 blockers/downregulators in the presence of an oxidized phospholipid so the effect of the test agent on the upregulated gene can be easily detected. However, the use of oxidized phospholipids is not required as agents can be generally screened for the ability to inhibit/block MKP-1 expression/activity.

It was also a discovery of this invention that administration of various synthetic phospholipids

In particular it was a discovery of this invention that 1,2-dimyristoyl-sn-glycero-3-phosphocholine (1,2-ditetradecanoyl-sn-glycero-3-phosphocholine) by itself was able to raise HDL levels, paraoxonase activity (a protective enzyme associated with HDL), was able to render HDL more effective in protecting LDL against oxidation and rendered LDL more resistant to oxidation by artery walls and dramatically reduced atherosclerosis in genetically engineered mice that develop atherosclerosis resembling human atherosclerosis. It is believed that administration of this and related lipids can also mitigate symptoms of an inflammatory condition or a pathology characterized by a chronic inflammatory condition or an acute inflammatory response.

Thus in one embodiment this invention provides methods of ameliorating one or more symptoms of atherosclerosis in a mammal. Preferred methods comprise administering to the mammal a phospholipid in an amount sufficient to ameliorate one or more symptoms of atherosclerosis. Similar methods can be used to mitigate or prevent a coronary complication associated with an acute phase response to an inflammation in a mammal, where said coronary complication is a symptom of atherosclerosis. These methods involve administering to a mammal having said acute phase response, or at risk for an acute phase response, a phospholipid in an amount sufficient to mitigate or prevent the coronary complication.

I. Assays for Agents that Modulate Oxidized Phospholipid Induced MKP-1 Expression.

As indicated above, in one aspect, this invention is premised on the discovery that oxidized phospholipids induce expression of MKP-1 and the upregulation/expression of MKP-1 produces an inflammatory response characteristic of plaque formation in atherosclerosis and/or rheumatoid arthritis or other inflammatory pathologies. Moreover, when such expression is inhibited in induced endothelial cells, the endothelial cells do not show induced monocyte binding or monocyte chemotaxis after exposure to oxidized phospholipids. Thus, agents that block the upregulation of MKP-1 by oxidized phospholipids are expected to be useful in ameliorating symptoms of atherosclerosis (e.g. plaque formation, monocyte binding, heart attack, stroke), rheumatoid arthritis, lupus, viral infections, and/or osteoporosis.

Accordingly, in one embodiment, this invention provides methods of screening for agents that modulate oxidized phospholipid-induced MKP-1 expression and hence one or more symptoms of atherosclerosis, one or more factors in the etiology of heart attack and/or stroke, and/or one or more symptoms of rheumatoid arthritis, lupus, viral infections, osteoporosis, etc. The methods involve detecting the expression level and/or activity level of an MKP-1 gene or gene product (e.g. MAP kinase phosphatase 1) in the presence of the agent(s) in question. Inhibition of expression of MKP-1 and/or inhibition of activity of MKP-1 polypeptides in the presence of the agent as compared to a negative control where the test agent is absent or at reduced concentration indicates that the agent ameliorates the response of MKP-1 and is potentially a good lead compound in the prophylaxis and/or treatment of atherosclerosis, cardiac pathologies, stroke, or rheumatoid arthritis, lupus, viral infections, osteoporosis. In certain preferred embodiments, the assay is done in a format where an oxidized phospholipid is used to upregulate MKP-1 and the agent is screened for the ability to inhibit the oxidized phospholipid-induced MKP-1 expression/activity.

In preferred embodiments, the assays involve contacting cells (e.g. in culture or in vivo) with one or more test agents and with an oxidized phospholipid (e.g. Ox-PAPC). The expression level of the MKP-1 gene is determined and a decrease in the level of expression induced by the oxidized phospholipid in cells treated with the test agent indicates that the test agent(s) ameliorate one or more symptoms of atherosclerosis as discussed above. The test agents can be administered before, simultaneously with, or after contacting the cell(s) with the oxidized phospholipid.

A single oxidized phospholipid can be used or combinations of different oxidized phospholipids can be used. Virtually any oxidized phospholipid can be used in this assay. Suitability of any particular phospholipid or combination of phospholipids can be readily determined by contacting a cell with the oxidized phospholipid(s) and determining whether or not the MKP-1 gene is upregulated as described herein.

Particularly preferred oxidized phospholipids include, but are not limited to oxidized forms of 1-palmitoyl-2-arachidonoyl-sn-glycero-3-phosphorylcholine (Ox-PAPC), 1-palmitoyl-2-oxovaleroyl-sn-glycero-3-phosphorylcholine (POVPC), 1-palrnitoyl-2-glutaroyl-sn-glycero-3-phosphorylcholine (PGPC),1-palmitoyl-2-epoxyisoprostane-sn-glycero-3-phosphorylcholine (PEIPC), oxidized 1-stearoyl-2-arachidonoyl-sn-glycero-3-phosphorylcholine (Ox-SAPC), 1-stearoyl-2-oxovaleroyl-sn-glycero-3-phosphorylcholine (SOVPC, 1-stearoyl-2-glutaroyl-sn-glycero-3-phosphorylcholine (SGPC), 1-stearoyl-2-epoxyisoprostane-sn-glycero-3-phosphorylcholine (SEIPC), 1-stearoyl-2-arachidonyl-sn-glycero-3-phosphorylethanolamine(Ox-SAPE), 1-stearoyl-2-oxovaleroyl-sn-glycero-3-phosphorylethanolamine(SOVPE), 1-stearoyl-2-glutaroyl-sn-glycero-3-phosphorylethanolamine(SGPE), 1-stearoyl-2-epoxyisoprostane-sn-glycero-3-phosphorylethanolamine(SEIPE), or related phospholipid oxidation products and the like.

Expression levels of a gene (e.g. ERP/MKP-1) can be altered by changes in the transcription of the gene product (i.e. transcription of mRNA), and/or by changes in translation of the gene product (i.e. translation of the protein), and/or by post-translational modification(s) (e.g. protein folding, glycosylation, etc.). Thus preferred assays of this invention include assaying for level of transcribed mRNA (or other nucleic acids derived from the MKP-1 gene), level of translated protein, activity of translated protein, etc. Examples of such approaches are described below.

A) Nucleic-Acid Based Assays.

1) Target Molecules.

Changes in MKP-1 expression level can be detected by measuring changes in MKP-1 mRNA and/or a nucleic acid derived from the mRNA (e.g. reverse-transcribed cDNA, etc.). In order to measure the MKP-1 expression level it is desirable to provide a nucleic acid sample for such analysis. In preferred embodiments the nucleic acid is found in or derived from a biological sample. The term “biological sample”, as used herein, refers to a sample obtained from an organism, from components (e.g., cells) of an organism, and/or from in vitro cell or tissue cultures. The sample may be of any biological tissue or fluid. Biological samples may also include organs or sections of tissues such as frozen sections taken for histological purposes.

The nucleic acid (e.g., mRNA or a nucleic acid derived from mRNA) is, in certain preferred embodiments, isolated from the sample according to any of a number of methods well known to those of skill in the art. Methods of isolating mRNA are well known to those of skill in the art. For example, methods of isolation and purification of nucleic acids are described in detail in by Tijssen ed., (1993) Chapter 3 of Laboratory Techniques in Biochemistry and Molecular Biology: Hybridization With Nucleic Acid Probes, Part I. Theory and Nucleic Acid Preparation, Elsevier, N.Y. and Tijssen ed.

In a preferred embodiment, the “total” nucleic acid is isolated from a given sample using, for example, an acid guanidinium-phenol-chloroform extraction method and polyA+mRNA is isolated by oligo dT column chromatography or by using (dT)n magnetic beads (see, e.g., Sambrook et al., Molecular Cloning: A Laboratory Manual (2nd ed.), Vols. 1-3, Cold Spring Harbor Laboratory, (1989), or Current Protocols in Molecular Biology, F. Ausubel et al., ed. Greene Publishing and Wiley-Interscience, New York (1987)).

Frequently, it is desirable to amplify the nucleic acid sample prior to assaying for expression level. Methods of amplifying nucleic acids are well known to those of skill in the art and include, but are not limited to polymerase chain reaction (PCR, see e.g., Innis, et al., (1990) PCR Protocols. A guide to Methods and Application. Academic Press, Inc. San Diego,), ligase chain reaction (LCR) (see Wu and Wallace (1989) Genomics 4: 560, Landegren et al. (1988) Science 241: 1077, and Barringer et al. (1990) Gene 89: 117, transcription amplification (Kwoh et al. (1989) Proc. Natl. Acad. Sci. USA_{—}86: 1173), self-sustained sequence replication (Guatelli et al. (1990) Proc. Nat. Acad. Sci. USA 87: 1874), dot PCR, and linker adapter PCR, etc.).

In a particularly preferred embodiment, where it is desired to quantify the transcription level (and thereby expression) of MKP-1 in a sample, the nucleic acid sample is one in which the concentration of the MKP-1 mRNA transcript(s), or the concentration of the nucleic acids derived from the MKP-1 mRNA transcript(s), is proportional to the transcription level (and therefore expression level) of that gene. Similarly, it is preferred that the hybridization signal intensity be proportional to the amount of hybridized nucleic acid. While it is preferred that the proportionality be relatively strict (e.g., a doubling in transcription rate results in a doubling in mRNA transcript in the sample nucleic acid pool and a doubling in hybridization signal), one of skill will appreciate that the proportionality can be more relaxed and even non-linear. Thus, for example, an assay where a 5 fold difference in concentration of the target mRNA results in a 3 to 6 fold difference in hybridization intensity is sufficient for most purposes.

Where more precise quantification is required appropriate controls can be run to correct for variations introduced in sample preparation and hybridization as described herein. In addition, serial dilutions of “standard” target nucleic acids (e.g., mRNAs) can be used to prepare calibration curves according to methods well known to those of skill in the art. Of course, where simple detection of the presence or absence of a transcript or large differences of changes in nucleic acid concentration is desired, no elaborate control or calibration is required.

In the simplest embodiment, the MKP-1-containing nucleic acid sample is the total mRNA or a total cDNA isolated and/or otherwise derived from a biological sample. The nucleic acid may be isolated from the sample according to any of a number of methods well known to those of skill in the art as indicated above.

2) Hybridization-Based Assays.

Using the known sequence of MKP-1 (see, e.g., GenBank Accession No: x68277) detecting and/or quantifying the MKP-1 transcript(s) can be routinely accomplished using nucleic acid hybridization techniques (see, e.g., Sambrook et al. supra). For example, one method for evaluating the presence, absence, or quantity of MKP-1 reverse-transcribed cDNA involves a “Southern Blot”. In a Southern Blot, the DNA (e.g., reverse-transcribed MKP-1 mRNA), typically fragmented and separated on an electrophoretic gel, is hybridized to a probe specific for MKP-1 (e.g. SEQ ID NO: 1). Comparison of the intensity of the hybridization signal from the MKP-1 probe with a “control” probe (e.g. a probe for a “housekeeping gene) provides an estimate of the relative expression level of the target nucleic acid.

Alternatively, the MKP-1 mRNA can be directly quantified in a Northern blot. In brief, the mRNA is isolated from a given cell sample using, for example, an acid guanidinium-phenol-chloroform extraction method. The mRNA is then electrophoresed to separate the mRNA species and the mRNA is transferred from the gel to a nitrocellulose membrane. As with the Southern blots, labeled probes are used to identify and/or quantify the target MKP-1 mRNA. Appropriate controls (e.g. probes to housekeeping genes) provide a reference for evaluating relative expression level.

An alternative means for determining the MKP-1 expression level is in situ hybridization. In situ hybridization assays are well known (e.g., Angerer (1987) Meth. Enzymol 152: 649). Generally, in situ hybridization comprises the following major steps: (1) fixation of tissue or biological structure to be analyzed; (2) prehybridization treatment of the biological structure to increase accessibility of target DNA, and to reduce nonspecific binding; (3) hybridization of the mixture of nucleic acids to the nucleic acid in the biological structure or tissue; (4) post-hybridization washes to remove nucleic acid fragments not bound in the hybridization and (5) detection of the hybridized nucleic acid fragments. The reagent used in each of these steps and the conditions for use vary depending on the particular application.

In some applications it is necessary to block the hybridization capacity of repetitive sequences. Thus, in some embodiments, tRNA, human genomic DNA, or Cot-1 DNA is used to block non-specific hybridization.

3) Amplifilcation-Based Assays.

In another embodiment, amplification-based assays can be used to measure MKP-1 expression (transcription) level. In such amplification-based assays, the target nucleic acid sequences (i.e., MKP-1 or fragments thereof) act as template(s) in amplification reaction(s) (e.g. Polymerase Chain Reaction (PCR) or reverse-transcription PCR (RT-PCR)). In a quantitative amplification, the amount of amplification product will be proportional to the amount of template (e.g., MKP-1 mRNA) in the original sample. Comparison to appropriate (e.g. healthy tissue or cells unexposed to the test agent) controls provides a measure of the MKP-1 transcript level.

Methods of “quantitative” amplification are well known to those of skill in the art. For example, quantitative PCR involves simultaneously co-amplifying a known quantity of a control sequence using the same primers. This provides an internal standard that may be used to calibrate the PCR reaction. Detailed protocols for quantitative PCR are provided in Innis et al. (1990) PCR Protocols, A Guide to Methods and Applications, Academic Press, Inc. N.Y.). One approach, for example, involves simultaneously co-amplifying a known quantity of a control sequence using the same primers as those used to amplify the target. This provides an internal standard that may be used to calibrate the PCR reaction.

One preferred internal standard is a synthetic AW106 cRNA. The AW106 cRNA is combined with RNA isolated from the sample according to standard techniques known to those of skill in the art. The RNA is then reverse transcribed using a reverse transcriptase to provide copy DNA. The cDNA sequences are then amplified (e.g., by PCR) using labeled primers. The amplification products are separated, typically by electrophoresis, and the amount of labeled nucleic acid (proportional to the amount of amplified product) is determined. The amount of mRNA in the sample is then calculated by comparison with the signal produced by the known AW106 RNA standard. Detailed protocols for quantitative PCR are provided in PCR Protocols, A Guide to Methods and Applications, Innis et al. (1990) Academic Press, Inc. N.Y. The known nucleic acid sequence(s) for MKP-1 are sufficient to enable one of skill to routinely select primers to amplify any portion of the gene.

4) Hybridization Formats and Optimization of Hybridization Conditions.

a) Array-Based Hybridization Formats.

In one embodiment, the methods of this invention can be utilized in array-based hybridization formats. Arrays are a multiplicity of different “probe” or “target” nucleic acids (or other compounds) attached to one or more surfaces (e.g., solid, membrane, or gel). In a preferred embodiment, the multiplicity of nucleic acids (or other moieties) is attached to a single contiguous surface or to a multiplicity of surfaces juxtaposed to each other.

In an array format a large number of different hybridization reactions can be run essentially “in parallel.” This provides rapid, essentially simultaneous, evaluation of a number of hybridizations in a single “experiment”. Methods of performing hybridization reactions in array based formats are well known to those of skill in the art (see, e.g., Pastinen (1997) Genome Res. 7: 606-614; Jackson (1996) Nature Biotechnology 14:1685; Chee (1995) Science 274: 610; WO 96/17958, Pinkel et al. (1998) Nature Genetics 20: 207-211).

Arrays, particularly nucleic acid arrays can be produced according to a wide variety of methods well known to those of skill in the art. For example, in a simple embodiment, “low density” arrays can simply be produced by spotting (e.g. by hand using a pipette) different nucleic acids at different locations on a solid support (e.g. a glass surface, a membrane, etc.).

This simple spotting, approach has been automated to produce high density spotted arrays (see, e.g., U.S. Pat. No. 5,807,522). This patent describes the use of an automated system that taps a microcapillary against a surface to deposit a small volume of a biological sample. The process is repeated to generate high density arrays.

Arrays can also be produced using oligonucleotide synthesis technology. Thus, for example; U.S. Pat. No. 5,143,854 and PCT Patent Publication Nos. WO 90/15070 and 92/10092 teach the use of light-directed combinatorial synthesis of high density oligonucleotide arrays. Synthesis of high density arrays is also described in U.S. Pat. Nos. 5,744,305, 5,800,992 and 5,445,934.

b) Other Hybridization Formats.

As indicated above a variety of nucleic acid hybridization formats are known to those skilled in the art. For example, common formats include sandwich assays and competition or displacement assays. Such assay formats are generally described in Hames and Higgins (1985) Nucleic Acid Hybridization, A Practical Approach, IRL Press; Gall and Pardue (1969) Proc. Natl. Acad. Sci. USA 63: 378-383; and John et al. (1969) Nature 223: 582-587.

Sandwich assays are commercially useful hybridization assays for detecting or isolating nucleic acid sequences. Such assays utilize a “capture” nucleic acid covalently immobilized to a solid support and a labeled “signal” nucleic acid in solution. The sample will provide the target nucleic acid. The “capture” nucleic acid and “signal” nucleic acid probe hybridize with the target nucleic acid to form a “sandwich” hybridization complex. To be most effective, the signal nucleic acid should not hybridize with the capture nucleic acid.

Typically, labeled signal nucleic acids are used to detect hybridization. Complementary nucleic acids or signal nucleic acids may be labeled by any one of several methods typically used to detect the presence of hybridized polynucleotides. The most common method of detection is the use of autoradiography with ³H, ¹²⁵I, ³⁵S, ¹⁴C, or ³²P-labelled probes or the like. Other labels include ligands that bind to labeled antibodies, fluorophores, chemiluminescent agents, enzymes, and antibodies which can serve as specific binding pair members for a labeled ligand.

Detection of a hybridization complex may require the binding of a signal generating complex to a duplex of target and probe polynucleotides or nucleic acids. Typically, such binding occurs through ligand and anti-ligand interactions as between a ligand-conjugated probe and an anti-ligand conjugated with a signal.

The sensitivity of the hybridization assays may be enhanced through use of a nucleic acid amplification system that multiplies the target nucleic acid being detected. Examples of such systems include the polymerase chain reaction (PCR) system and the ligase chain reaction (LCR) system. Other methods recently described in the art are the nucleic acid sequence based amplification (NASBAO, Cangene, Mississauga, Ontario) and Q Beta Replicase systems.

c) Optimization of Hybridization Conditions.

Nucleic acid hybridization simply involves providing a denatured probe and target nucleic acid under conditions where the probe and its complementary target can form stable hybrid duplexes through complementary base pairing. The nucleic acids that do not form hybrid duplexes are then washed away leaving the hybridized nucleic acids to be detected, typically through detection of an attached detectable label. It is generally recognized that nucleic acids are denatured by increasing the temperature or decreasing the salt concentration of the buffer containing the nucleic acids, or in the addition of chemical agents, or the raising of the pH. Under low stringency conditions (e.g., low temperature and/or high salt and/or high target concentration) hybrid duplexes (e.g., DNA:DNA, RNA:RNA, or RNA:DNA) will form even where the annealed sequences are not perfectly complementary. Thus specificity of hybridization is reduced at lower stringency. Conversely, at higher stringency (e.g., higher temperature or lower salt) successful hybridization requires fewer mismatches.

One of skill in the art will appreciate that hybridization conditions may be selected to provide any degree of stringency. In a preferred embodiment, hybridization is performed at low stringency to ensure hybridization and then subsequent washes are performed at higher stringency to eliminate mismatched hybrid duplexes. Successive washes may be performed at increasingly higher stringency (e.g., down to as low as 0.25× SSPE at 37° C. to 70° C.) until a desired level of hybridization specificity is obtained. Stringency can also be increased by addition of agents such as formamide. Hybridization specificity may be evaluated by comparison of hybridization to the test probes with hybridization to the various controls that can be present.

In general, there is a tradeoff between hybridization specificity (stringency) and signal intensity. Thus, in a preferred embodiment, the wash is performed at the highest stringency that produces consistent results and that provides a signal intensity greater than approximately 10% of the background intensity. Thus, in a preferred embodiment, the hybridized array may be washed at successively higher stringency solutions and read between each wash. Analysis of the data sets thus produced will reveal a wash stringency above which the hybridization pattern is not appreciably altered and which provides adequate signal for the particular probes of interest.

In a preferred embodiment, background signal is reduced by the use of a blocking reagent (e.g., tRNA, sperm DNA, cot-1 DNA, etc.) during the hybridization to reduce non-specific binding. The use of blocking agents in hybridization is well known to those of skill in the art (see, e.g., Chapter 8 in P. Tijssen, supra.)

Methods of optimizing hybridization conditions are well known to those of skill in the art (see, e.g., Tijssen (1993) Laboratory Techniques in Biochemistry and Molecular Biology, Vol. 24: Hybridization With Nucleic Acid Probes, Elsevier, N.Y.).

Optimal conditions are also a function of the sensitivity of label (e.g., fluorescence) detection for different combinations of substrate type, fluorochrome, excitation and emission bands, spot size and the like. Low fluorescence background surfaces can be used (see, e.g., Chu (1992) Electrophoresis 13:105-114). The sensitivity for detection of spots (“target elements”) of various diameters on the candidate surfaces can be readily determined by, e.g., spotting a dilution series of fluorescently end labeled DNA fragments. These spots are then imaged using conventional fluorescence microscopy. The sensitivity, linearity, and dynamic range achievable from the various combinations of fluorochrome and solid surfaces (e.g., glass, fused silica, etc.) can thus be determined. Serial dilutions of pairs of fluorochrome in known relative proportions can also be analyzed. This determines the accuracy with which fluorescence ratio measurements reflect actual fluorochrome ratios over the dynamic range permitted by the detectors and fluorescence of the substrate upon which the probe has been fixed.

d) Labeling and Detection of Nucleic Acids.

The probes used herein for detection of MKP-1 expression levels can be full length or less than the full length of the MKP-1 mRNA. Shorter probes are empirically tested for specificity. Preferred probes are sufficiently long so as to specifically hybridize with the MKP-1 target nucleic acid(s) under stringent conditions. The preferred size range is from about 10 bases to the length of the MKP-1 mRNA, preferably from about 15 or 20 bases to the length of the MKP-1 mRNA, more preferably from about 30 bases to the length of the MKP-1 mRNA, and most preferably from about 40 bases to the length of the MKP-1 mRNA.

The probes are typically labeled, with a detectable label. Detectable labels suitable for use in the present invention include any composition detectable by spectroscopic, photochemical, biochemical, immunochemical, electrical, optical or chemical means. Useful labels in the present invention include biotin for staining with labeled streptavidin conjugate, magnetic beads (e.g., Dynabeads™), fluorescent dyes (e.g., fluorescein, texas red, rhodamine, green fluorescent protein, and the like, see, e.g., Molecular Probes, Eugene, Oreg., USA), radiolabels (e.g., ³H, ¹²⁵I, ³⁵S, ¹⁴C, or ³²P), enzymes (e.g., horse radish peroxidase, alkaline phosphatase and others commonly used in an ELISA), and colorimetric labels such as colloidal gold (e.g., gold particles in the 40-80 nm diameter size range scatter green light with high efficiency) or colored glass or plastic (e.g., polystyrene, polypropylene, latex, etc.) beads. Patents teaching the use of such labels include U.S. Pat. Nos. 3,817,837; 3,850,752; 3,939,350; 3,996,345; 4,277,437; 4,275,149; and 4,366,241.

A fluorescent label is preferred because it provides a very strong signal with low background. It is also optically detectable at high resolution and sensitivity through a quick scanning procedure. The nucleic acid samples can all be labeled with a single label, e.g., a single fluorescent label. Alternatively, in another embodiment, different nucleic acid samples can be simultaneously hybridized where each nucleic acid sample has a different label. For instance, one target could have a green fluorescent label and a second target could have a red fluorescent label. The scanning step will distinguish sites of binding of the red label from those binding the green fluorescent label. Each nucleic acid sample (target nucleic acid) can be analyzed independently from one another.

Suitable chromogens which can be employed include those molecules and compounds which absorb light in a distinctive range of wavelengths so that a color can be observed or, alternatively, which emit light when irradiated with radiation of a particular wave length or wave length range, e.g., fluorescers.

Desirably, fluorescent labels should absorb light above about 300 nm, preferably about 350 nm, and more preferably above about 400 nm, usually emitting at wavelengths greater than about 10 nm higher than the wavelength of the light absorbed. It should be noted that the absorption and emission characteristics of the bound dye can differ from the unbound dye. Therefore, when referring to the various wavelength ranges and characteristics of the dyes, it is intended to indicate the dyes as employed and not the dye which is unconjugated and characterized in an arbitrary solvent.

Fluorescers are generally preferred because by irradiating a fluorescer with light, one can obtain a plurality of emissions. Thus, a single label can provide for a plurality of measurable events.

Detectable signal can also be provided by chemiluminescent and bioluminescent sources. Chemiluminescent sources include a compound which becomes electronically excited by a chemical reaction and can then emit light which serves as the detectable signal or donates energy to a fluorescent acceptor. Alternatively, luciferins can be used in conjunction with luciferase or lucigenins to provide bioluminescence.

Spin labels are provided by reporter molecules with an unpaired electron spin which can be detected by electron spin resonance (ESR) spectroscopy. Exemplary spin labels include organic free radicals, transitional metal complexes, particularly vanadium, copper, iron, and manganese, and the like. Exemplary spin labels include nitroxide free radicals.

The label may be added to the target (sample) nucleic acid(s) prior to, or after the hybridization. So called “direct labels” are detectable labels that are directly attached to or incorporated into the target (sample) nucleic acid prior to hybridization. In contrast, so called “indirect labels” are joined to the hybrid duplex after hybridization. Often, the indirect label is attached to a binding moiety that has been attached to the target nucleic acid prior to the hybridization. Thus, for example, the target nucleic acid may be biotinylated before the hybridization. After hybridization, an avidin-conjugated fluorophore will bind the biotin bearing hybrid duplexes providing a label that is easily detected. For a detailed review of methods of labeling nucleic acids and detecting labeled hybridized nucleic acids see Laboratory Techniques in Biochemistry and Molecular Biology, Vol. 24: Hybridization With Nucleic Acid Probes, P. Tijssen, ed. Elsevier, N.Y., (1993)).

Fluorescent labels are easily added during an in vitro transcription reaction. Thus, for example, fluorescein labeled UTP and CTP can be incorporated into the RNA produced in an in vitro transcription.

The labels can be attached directly or through a linker moiety. In general, the site of label or linker-label attachment is not limited to any specific position. For example, a label may be attached to a nucleoside, nucleotide, or analogue thereof at any position that does not interfere with detection or hybridization as desired. For example, certain Label-ON Reagents from Clontech (Palo Alto, Calif.) provide for labeling interspersed throughout the phosphate backbone of an oligonucleotide and for terminal labeling at the 3′ and 5′ ends. As shown for example herein, labels can be attached at positions on the ribose ring or the ribose can be modified and even eliminated as desired. The base moieties of useful labeling reagents can include those that are naturally occurring or modified in a manner that does not interfere with the purpose to which they are put. Modified bases include but are not limited to 7-deaza A and G, 7-deaza-8-aza A and G, and other heterocyclic moieties.

It will be recognized that fluorescent labels are not to be limited to single species organic molecules, but include inorganic molecules, multi-molecular mixtures of organic and/or inorganic molecules, crystals, heteropolymers, and the like. Thus, for example, CdSe-CdS core-shell nanocrystals enclosed in a silica shell can be easily derivatized for coupling to a biological molecule (Bruchez et al. (1998) Science, 281: 2013-2016). Similarly, highly fluorescent quantum dots (zinc sulfide-capped cadmium selenide) have been covalently coupled to biomolecules for use in ultrasensitive biological detection (Warren and Nie (1998) Science, 281: 2016-2018).

B) Polypeptide-Based Assays.

1) Assay Formats.

In addition to, or in alternative to, the detection of MKP-1 nucleic acid expression level(s), alterations in expression of MKP-1 can be detected and/or quantified by detecting and/or quantifying the amount and/or activity of translated MKP-1 polypeptide.

2) Detection of Expressed Protein.

The polypeptide(s) encoded by the MKP-1 can be detected and quantified by any of a number of methods well known to those of skill in the art. These may include analytic biochemical methods such as electrophoresis, capillary electrophoresis, high performance liquid chromatography (HPLC), thin layer chromatography (TLC), hyperdiffusion chromatography, and the like, or various immunological methods such as immunohistochemistry, fluid or gel precipitin reactions, immunodiffusion (single or double), immunoelectrophoresis, radioimmunoassay (RIA), enzyme-linked immunosorbent assays (ELISAs), immunofluorescent assays, western blotting, and the like.

In one preferred embodiment, the MKP-1 polypeptide(s) are detected/quantified in an electrophoretic protein separation (e.g. a 1- or 2-dimensional electrophoresis). Means of detecting proteins using electrophoretic techniques are well known to those of skill in the art (see generally, R. Scopes (1982) Protein Purification, Springer-Verlag, N.Y.; Deutscher, (1990) Methods in Enzymology Vol. 182: Guide to Protein Purification, Academic Press, Inc., N.Y.).

In another preferred embodiment the MKP-1 polypeptide(s) are detected/quantified immunohistochemically. Immunohistochemical methods typically utilize a labeled anti-MKP-1 antibody to label and thereby quantify expressed MKP-1 polypeptide. Immunohistochemical methods for the detection/quantification of MKP-1 are well known to those of skill in the art (see, e.g., (1997) J. Biol. Chem. 272: 16917-16923.).

In still another preferred embodiment, Western blot (immunoblot) analysis is used to detect and quantify the presence of polypeptide(s) of this invention in the sample. This technique generally comprises separating sample proteins by gel electrophoresis on the basis of molecular weight, transferring the separated proteins to a suitable solid support, (such as a nitrocellulose filter, a nylon filter, or derivatized nylon filter), and incubating the sample with the antibodies that specifically bind the target polypeptide(s).

The antibodies specifically bind to the target polypeptide(s) and may be directly labeled or alternatively may be subsequently detected using labeled antibodies (e.g., labeled sheep anti-mouse antibodies) that specifically bind to the a domain of the antibody.

In preferred embodiments, the MKP-1 polypeptide(s) are detected using an immunoassay. As used herein, an immunoassay is an assay that utilizes an antibody to specifically bind to the analyte (e.g., the target polypeptide(s)). The immunoassay is thus characterized by detection of specific binding of a polypeptide of this invention to an antibody as opposed to the use of other physical or chemical properties to isolate, target, and quantify the analyte.

Any of a number of well recognized immunological binding assays (see, e.g., U.S. Pat. Nos. 4,366,241; 4,376,110; 4,517,288; and 4,837,168) are well suited to detection or quantification of the polypeptide(s) identified herein. For a review of the general immunoassays, see also Asai (1993) Methods in Cell Biology Volume 37: Antibodies in Cell Biology, Academic Press, Inc. New York; Stites & Terr (1991) Basic and Clinical Immunology 7th Edition.

Immunological binding assays (or immunoassays) typically utilize a “capture agent” to specifically bind to and often immobilize the analyte (MKP-1 polypeptide). In preferred embodiments, the capture agent is an antibody.

Immunoassays also often utilize a labeling agent to specifically bind to and label the binding complex formed by the capture agent and the analyte. The labeling agent may itself be one of the moieties comprising the antibody/analyte complex. Thus, the labeling agent may be a labeled polypeptide or a labeled antibody that specifically recognizes the already bound target polypeptide. Alternatively, the labeling agent may be a third moiety, such as another antibody, that specifically binds to the capture agent /polypeptide complex.

Other proteins capable of specifically binding immunoglobulin constant regions, such as protein A or protein G may also be used as the label agent. These proteins are normal constituents of the cell walls of streptococcal bacteria. They exhibit a strong non-immunogenic reactivity with immunoglobulin constant regions from a variety of species (see, generally Kronval, et al. (1973) J. Immunol., 111: 1401-1406, and Akerstrom (1985) J. Immunol., 135: 2589-2542).

Preferred immunoassays for detecting the target polypeptide(s) are either competitive or noncompetitive. Noncompetitive immunoassays are assays in which the amount of captured analyte is directly measured. In one preferred “sandwich” assay, for example, the capture agents (antibodies) can be bound directly to a solid substrate where they are immobilized. These immobilized antibodies then capture the target polypeptide present in the test sample. The target polypeptide thus immobilized is then bound by a labeling agent, such as a second antibody bearing a label.

In competitive assays, the amount of analyte (MKP-1 polypeptide) present in the sample is measured indirectly by measuring the amount of an added (exogenous) analyte displaced (or competed away) from a capture agent (antibody) by the analyte present in the sample. In one competitive assay, a known amount of, in this case, labeled polypeptide is added to the sample and the sample is then contacted with a capture agent. The amount of labeled polypeptide bound to the antibody is inversely proportional to the concentration of target polypeptide present in the sample.

In one particularly preferred embodiment, the antibody is immobilized on a solid substrate. The amount of target polypeptide bound to the antibody may be determined either by measuring the amount of target polypeptide present in an polypeptide/antibody complex, or alternatively by measuring the amount of remaining uncomplexed polypeptide.

The immunoassay methods of the present invention include an enzyme immunoassay (EIA) which utilizes, depending on the particular protocol employed, unlabeled or labeled (e.g., enzyme-labeled) derivatives of polyclonal or monoclonal antibodies or antibody fragments or single-chain antibodies that bind MKP-1 polypeptide(s), either alone or in combination. In the case where the antibody that binds MKP-1 polypeptide is not labeled, a different detectable marker, for example, an enzyme-labeled antibody capable of binding to the monoclonal antibody which binds the MKP-1 polypeptide, may be employed. Any of the known modifications of EIA, for example, enzyme-linked immunoabsorbent assay (ELISA), may also be employed. As indicated above, also contemplated by the present invention are immunoblotting immunoassay techniques such as western blotting employing an enzymatic detection system.

The immunoassay methods of the present invention may also be other known immunoassay methods, for example, fluorescent immunoassays using antibody conjugates or antigen conjugates of fluorescent substances such as fluorescein or rhodamine, latex agglutination with antibody-coated or antigen-coated latex particles, haemagglutination with antibody-coated or antigen-coated red blood corpuscles, and immunoassays employing an avidin-biotin or strepavidin-biotin detection systems, and the like.

The particular parameters employed in the immunoassays of the present invention can vary widely depending on various factors such as the concentration of antigen in the sample, the nature of the sample, the type of immunoassay employed and the like. Optimal conditions can be readily established by those of ordinary skill in the art. In certain embodiments, the amount of antibody that binds MKP-1 polypeptides is typically selected to give 50% binding of detectable marker in the absence of sample. If purified antibody is used as the antibody source, the amount of antibody used per assay will generally range from about 1 ng to about 100 ng. Typical assay conditions include a temperature range of about 4° C. to about 45° C., preferably about 25° C. to about 37° C., and most preferably about 25° C., a pH value range of about 5 to 9, preferably about 7, and an ionic strength varying from that of distilled water to that of about 0.2M sodium chloride, preferably about that of 0.15M sodium chloride. Times will vary widely depending upon the nature of the assay, and generally range from about 0.1 minute to about 24 hours. A wide variety of buffers, for example PBS, may be employed, and other reagents such as salt to enhance ionic strength, proteins such as serum albumins, stabilizers, biocides and non-ionic detergents may also be included.

The assays of this invention are scored (as positive or negative or quantity of target polypeptide) according to standard methods well known to those of skill in the art. The particular method of scoring will depend on the assay format and choice of label. For example, a Western Blot assay can be scored by visualizing the colored product produced by the enzymatic label. A clearly visible colored band or spot at the correct molecular weight is scored as a positive result, while the absence of a clearly visible spot or band is scored as a negative. The intensity of the band or spot can provide a quantitative measure of target polypeptide concentration.

Antibodies for use in the various immunoassays described herein, are commercially available or can be produced as described below.

3) Detection of MKP-1 Activity.

Mitogen-activated protein (MAP) kinase phosphatase-1 (MKP-1) is a dual-specificity protein phosphatase encoded by an immediate-early gene responsive to growth factors and stress. The MKP-1 protein selectively inactivates MAP kinases in vitro by dephosphorylation of the regulatory Thr and Tyr residues (Scimeca et al. (1997) Oncogene 15(6): 717-725 and references therein). Thus, agents can be screened for the ability to inhibit MKP-1 mediated dephosphorylation of a target residue. Methods of assaying MKP-1 for dephosphorylation activity are known to those of skill in the art (see, e.g., Duff et al. (1995) 270: 13 7161-7166 and references therein.)

It was a discovery of this invention that MKP-1, in response to oxidized phospholipids, is upregulated and induces an “inflammation” response in vascular endothelium similar to that observed in atherosclerosis. Characteristics of this response include, but are not limited to monocyte binding and a monocyte chemotactic response. Methods of assaying mononcyte binding and/or a chemotactic response are well known to those of skill in the art (see, e.g. Berliner et al. (1990) J. Clinical Invest., 85(4): 1260-1266; Navab et al. (1997) J. Clin. Invest. 99(12): 3043; Navab et al. (1997) J. Clin. Invest. 99(8):2005-2019) and are illustrated in the Examples provided herein.

4) Antibodies to MKP-1 Polypeptides.

Either polyclonal or monoclonal antibodies may be used in the immunoassays of the invention described herein. Polyclonal antibodies are preferably raised by multiple injections (e.g. subcutaneous or intramuscular injections) of substantially pure polypeptides or antigenic polypeptides into a suitable non-human mammal. The antigenicity of the target peptides can be determined by conventional techniques to determine the magnitude of the antibody response of an animal that has been immunized with the peptide. Generally, the peptides that are used to raise antibodies for use in the methods of this invention should generally be those which induce production of high titers of antibody with relatively high affinity for target polypeptides encoded by MKP-1 genes.

If desired, the immunizing peptide may be coupled to a carrier protein by conjugation using techniques that are well-known in the art. Such commonly used carriers which are chemically coupled to the peptide include keyhole limpet hemocyanin (KLH), thyroglobulin, bovine serum albumin (BSA), and tetanus toxoid. The coupled peptide is then used to immunize the animal (e.g. a mouse or a rabbit).

The antibodies are then obtained from blood samples taken from the mammal. The techniques used to develop polyclonal antibodies are known in the art (see, e.g., Methods of Enzymology, “Production of Antisera With Small Doses of Immunogen: Multiple Intradermal Injections”, Langone, et al. eds. (Acad. Press, 1981)). Polyclonal antibodies produced by the animals can be further purified, for example, by binding to and elution from a matrix to which the peptide to which the antibodies were raised is bound. Those of skill in the art will know of various techniques common in the immunology arts for purification and/or concentration of polyclonal antibodies, as well as monoclonal antibodies see, for example, Coligan, et al. (1991) Unit 9, Current Protocols in Immunology, Wiley Interscience).

Preferably, however, the antibodies produced will be monoclonal antibodies (“mAb's”). For preparation of monoclonal antibodies, immunization of a mouse or rat is preferred. The term “antibody” as used in this invention includes intact molecules as well as fragments thereof, such as, Fab and F(ab′)², and/or single-chain antibodies (e.g. scFv) which are capable of binding an epitopic determinant. Also, in this context, the term “mab's of the invention” refers to monoclonal antibodies with specificity for a polypeptide encoded by a MKP-1 polypeptide.

The general method used for production of hybridomas secreting mAbs is well known (Kohler and Milstein (1975) Nature, 256:495). Briefly, as described by Kohler and Milstein the technique comprised isolating lymphocytes from regional draining lymph nodes of five separate cancer patients with either melanoma, teratocarcinoma or cancer of the cervix, glioma or lung, (where samples were obtained from surgical specimens), pooling the cells, and fusing the cells with SHFP-1. Hybridomas were screened for production of antibody which bound to cancer cell lines. Confirmation of specificity among mAb's can be accomplished using relatively routine screening techniques (such as the enzyme-linked immunosorbent assay, or “ELISA”) to determine the elementary reaction pattern of the mAb of interest.

Antibodies fragments, e.g. single chain antibodies (scFv or others), can also be produced/selected using phage display technology. The ability to express antibody fragments on the surface of viruses that infect bacteria (bacteriophage or phage) makes it possible to isolate a single binding antibody fragment, e.g., from a library of greater than 10¹⁰ nonbinding clones. To express antibody fragments on the surface of phage (phage display), an antibody fragment gene is inserted into the gene encoding a phage surface protein (e.g., pIII) and the antibody fragment-pIII fusion protein is displayed on the phage surface (McCafferty et al. (1990) Nature, 348: 552-554; Hoogenboom et al. (1991) Nucleic Acids Res. 19: 4133-4137).

Since the antibody fragments on the surface of the phage are functional, phage bearing antigen binding antibody fragments can be separated from non-binding phage by antigen affinity chromatography (McCafferty et al. (1990) Nature, 348: 552-554). Depending on the affinity of the antibody fragment, enrichment factors of 20 fold −1,000,000 fold are obtained for a single round of affinity selection. By infecting bacteria with the eluted phage, however, more phage can be grown and subjected to another round of selection. In this way, an enrichment of 1000 fold in one round can become 1,000,000 fold in two rounds of selection (McCafferty et al. (1990) Nature, 348: 552-554). Thus even when enrichments are low (Marks et al. (1991) J. Mol. Biol. 222: 581-597), multiple rounds of affinity selection can lead to the isolation of rare phage. Since selection of the phage antibody library on antigen results in enrichment, the majority of clones bind antigen after as few as three to four rounds of selection. Thus only a relatively small number of clones (several hundred) need to be analyzed for binding to antigen.

Human antibodies can be produced without prior immunization by displaying very large and diverse V-gene repertoires on phage (Marks et al. (1991) J. Mol. Biol. 222: 581-597). In one embodiment natural V_{H} and V_{L} repertoires present in human peripheral blood lymphocytes are were isolated from unimmunized donors by PCR. The V-gene repertoires were spliced together at random using PCR to create a scFv gene repertoire which is was cloned into a phage vector to create a library of 30 million phage antibodies (ld.). From this single “naive” phage antibody library, binding antibody fragments have been isolated against more than 17 different antigens, including haptens, polysaccharides and proteins (Marks et al. (1991) J. Mol. Biol. 222: 581-597; Marks et al. (1993). Bio/Technology. 10: 779-783; Griffiths et al. (1993) EMBO J. 12: 725-734; Clackson et al. (1991) Nature. 352: 624-628). Antibodies have been produced against self proteins, including human thyroglobulin, immunoglobulin, tumor necrosis factor and CEA (Griffiths et al. (1993) EMBO J. 12: 725-734). It is also possible to isolate antibodies against cell surface antigens by selecting directly on intact cells. The antibody fragments are highly specific for the antigen used for selection and have affinities in the 1:M to 100 nM range (Marks et al. (1991) J. Mol. Biol. 222: 581-597; Griffiths et al. (1993) EMBO J. 12: 725-734). Larger phage antibody libraries result in the isolation of more antibodies of higher binding affinity to a greater proportion of antigens.

It will also be recognized that antibodies can be prepared by any of a number of commercial services (e.g., Berkeley antibody laboratories, Bethyl Laboratories, Anawa, Eurogenetec, etc.). In addition MKP-1 antibodies are commercially available (see, e.g., Santa Cruz Biotechnology, Santa Cruz, USA).

C) Evaluating Changes in MKP-1 Expression.

In one embodiment, in the assays of this invention, a test agent is scored as positive when it reduces and/or eliminates upregulation of MKP-1 in response to an oxidized phospholipid. This can be by inhibition of transcription or translation or by diminution of the activity of the translated MKP-1 polypeptide. The assay is scored as positive when the oxidized phospholipid induced expression in the presence of the test agent(s) is detectably lower than the oxidized phospholipid-induced expression in the absence of the test agent or in an assay where the test agent is present at a lower concentration. The detectable difference is preferably a statistically significant difference, e.g. at a confidence level of 80% or better, preferably 90% or better, more preferably 95% or better, and most preferably 98% or 99% or better.

The comparison between test cells (cells contacted with the test agent(s)) and control cells (e.g. cells not contacted with the test agent(s)) can be a direct comparison, e.g. in simultaneously or sequentially performed experiments. Alternatively, the comparison can be an “indirect” comparison, i.e. with data previously obtained at a different time, and/or in a different set of experiments, and/or with data obtained by a different party.

D) Assay Optimization.

The assays of this invention have immediate utility in screening for agents that modulate the MKP-1 expression of a cell, tissue or organism. The assays of this invention can be optimized for use in particular contexts, depending, for example, on the source and/or nature of the biological sample and/or the particular test agents, and/or the analytic facilities available. Thus, for example, optimization can involve determining optimal conditions for binding assays, optimum sample processing conditions (e.g. preferred PCR conditions), hybridization conditions that maximize signal to noise, protocols that improve throughput, etc. In addition, assay formats can be selected and/or optimized according to the availability of equipment and/or reagents. Thus, for example, where commercial antibodies or ELISA kits are available it may be desired to assay protein concentration. Conversely, where it is desired to screen for modulators that alter transcription the MKP-1 gene, nucleic acid based assays are preferred.

Routine selection and optimization of assay formats is well known to those of ordinary skill in the art.

II. Prescreening for Agents that Bind MKP-1 Polypeptides and/or Nucleic Acids, or Agents that Bind to a Receptor that is Required for the Induction of MKP-1 Polypeptides and/or Nucleic Acids.

In certain embodiments it is desired to pre-screen test agents for the ability to interact with (e.g. specifically bind to) an MKP-1 nucleic acid or polypeptide. Specifically binding test agents are more likely to interact with and thereby modulate MKP-1 expression and/or activity. Thus, in some preferred embodiments, the test agent(s) are pre-screened for binding to MKP-1 nucleic acids or to MKP-1 proteins before performing the more complex assays described above.

In one embodiment, such pre-screening is accomplished with simple binding assays. Means of assaying for specific binding or the binding affinity of a particular ligand for a nucleic acid or for a protein are well known to those of skill in the art. In preferred binding assays, the MKP-1 protein or nucleic acid is immobilized and exposed to a test agent (which can be labeled), or alternatively, the test agent(s) are immobilized and exposed to an MKP-1 protein or to a MKP-1 nucleic acid (which can be labeled). The immobilized moiety is then washed to remove any unbound material and the bound test agent or bound MKP-1 nucleic acid or protein is detected (e.g. by detection of a label attached to the bound molecule). The amount of immobilized label is proportional to the degree of binding between the MKP-1 protein or nucleic acid and the test agent.

III. High Throughput Screening

The assays of this invention are also amenable to “high-throughput” modalities. Conventionally, new chemical entities with useful properties (e.g., modulation of oxidized phospholipid-induced expression or activity of MKP-1) are generated by identifying a chemical compound (called a “lead compound”) with some desirable property or activity, creating variants of the lead compound, and evaluating the property and activity of those variant compounds. However, the current trend is to shorten the time scale for all aspects of drug discovery. Because of the ability to test large numbers quickly and efficiently, high throughput screening (HTS) methods are replacing conventional lead compound identification methods.

In one preferred embodiment, high throughput screening methods involve providing a library containing a large number of compounds (candidate compounds) potentially having the desired activity. Such “combinatorial chemical libraries” are then screened in one or more assays, as described herein, to identify those library members (particular chemical species or subclasses) that display a desired characteristic activity. The compounds thus identified can serve as conventional “lead compounds” or can themselves be used as potential or actual therapeutics.

A) Combinatorial Chemical Libraries.

Recently, attention has focused on the use of combinatorial chemical libraries to assist in the generation of new chemical compound leads. A combinatorial chemical library is a collection of diverse chemical compounds generated by either chemical synthesis or biological synthesis by combining a number of chemical “building blocks” such as reagents. For example, a linear combinatorial chemical library such as a polypeptide library is formed by combining a set of chemical building blocks called amino acids in every possible way for a given compound length (i.e., the number of amino acids in a polypeptide compound). Millions of chemical compounds can be synthesized through such combinatorial mixing of chemical building blocks. For example, one commentator has observed that the systematic, combinatorial mixing of 100 interchangeable chemical building blocks results in the theoretical synthesis of 100 million tetrameric compounds or 10 billion pentameric compounds (Gallop et al. (1994) 37(9): 1233-1250).

Preparation and screening of combinatorial chemical libraries is well known to those of skill in the art. Such combinatorial chemical libraries include, but are not limited to, peptide libraries (see, e.g., U.S. Pat. No. 5,010,175, Furka (1991) Int. J. Pept. Prot. Res., 37: 487-493, Houghton et al. (1991) Nature, 354: 84-88). Peptide synthesis is by no means the only approach envisioned and intended for use with the present invention. Other chemistries for generating chemical diversity libraries can also be used. Such chemistries include, but are not limited to: peptoids (PCT Publication No WO 91/19735, Dec. 26, 1991), encoded peptides (PCT Publication WO 93/20242, Oct. 14, 1993), random bio-oligomers (PCT Publication WO 92/00091, Jan. 9, 1992), benzodiazepines (U.S. Pat. No. 5,288,514), diversomers such as hydantoins, benzodiazepines and dipeptides (Hobbs et al., (1993) Proc. Nat. Acad. Sci. USA 90: 6909-6913), vinylogous polypeptides (Hagihara et al. (1992) J. Amer. Chem. Soc. 114: 6568), nonpeptidal peptidomimetics with a Beta-D-Glucose scaffolding (Hirschmann et al., (1992) J. Amer. Chem. Soc. 114: 9217-9218), analogous organic syntheses of small compound libraries (Chen et al. (1994) J. Amer. Chem. Soc. 116: 2661), oligocarbamates (Cho, et al., (1993) Science 261:1303), and/or peptidyl phosphonates (Campbell et al., (1994) J. Org. Chem. 59: 658). See, generally, Gordon et al., (1994) J. Med. Chem. 37:1385, nucleic acid libraries (see, e.g., Strategene, Corp.), peptide nucleic acid libraries (see, e.g., U.S. Pat. No. 5,539,083) antibody libraries (see, e.g., Vaughn et al. (1996) Nature Biotechnology, 14(3): 309-314), and PCT/US96/10287), carbohydrate libraries (see, e.g., Liang et al. (1996) Science, 274: 1520-1522, and U.S. Pat. No. 5,593,853), and small organic molecule libraries (see, e.g., benzodiazepines, Baum (1993) C&EN, January 18, page 33, isoprenoids U.S. Pat. No. 5,569,588, thiazolidinones and metathiazanones U.S. Pat. No. 5,549,974, pyrrolidines U.S. Pat. Nos. 5,525,735 and 5,519,134, morpholino compounds U.S. Pat. No. 5,506,337, benzodiazepines 5,288,514, and the like).

Devices for the preparation of combinatorial libraries are commercially available (see, e.g., 357 MPS, 390 MPS, Advanced Chem Tech, Louisville Ky., Symphony, Rainin, Woburn, Mass., 433A Applied Biosystems, Foster City, Calif., 9050 Plus, Millipore, Bedford, Mass.).

A number of well known robotic systems have also been developed for solution phase chemistries. These systems include automated workstations like the automated synthesis apparatus developed by Takeda Chemical Industries, LTD. (Osaka, Japan) and many robotic systems utilizing robotic arms (Zymate II, Zymark Corporation, Hopkinton, Mass.; Orca, Hewlett-Packard, Palo Alto, Calif.) which mimic the manual synthetic operations performed by a chemist. Any of the above devices are suitable for use with the present invention. The nature and implementation of modifications to these devices (if any) so that they can operate as discussed herein will be apparent to persons skilled in the relevant art. In addition, numerous combinatorial libraries are themselves commercially available (see, e.g., ComGenex, Princeton, N.J., Asinex, Moscow, RU, Tripos, Inc., St. Louis, Mo., ChemStar, Ltd, Moscow, RU, 3D Pharmaceuticals, Exton, Pa., Martek Biosciences, Columbia, Md., etc.).

B) High Throughput Assays of Chemical Libraries.

Any of the assays for that modulate expression of MKP-1 or that alter the binding specificity and/or activity of MKP-1 polypeptides are amenable to high throughput screening. As described above, having determined that oxidized phospholipid-induced MKP-1 expression features characteristic of atherosclerosis, cardiac diseases, stroke or rheumatoid arthritis, inhibitors of the MKP-1 response to oxidized phospholipids are likely to ameliorate symptoms of these conditions, thus motivating the assays described herein. Such assays are amenable to high-throughput modalities.

High throughput assays for the presence, absence, or quantification of particular nucleic acids or protein products are well known to those of skill in the art. Similarly, binding assays are similarly well known. Thus, for example, U.S. Pat. No. 5,559,410 discloses high throughput screening methods for proteins, U.S. Pat. No. 5,585,639 discloses high throughput screening methods for nucleic acid binding (i.e., in arrays), while U.S. Pat. Nos. 5,576,220 and 5,541,061 disclose high throughput methods of screening for ligand/antibody binding.

In addition, high throughput screening systems are commercially available (see, e.g., Zymark Corp., Hopkinton, Mass.; Air Technical Industries, Mentor, Ohio; Beckman Instruments, Inc. Fullerton, Calif.; Precision Systems, Inc., Natick, Mass., etc.). These systems typically automate entire procedures including all sample and reagent pipetting, liquid dispensing, timed incubations, and final readings of the microplate in detector(s) appropriate for the assay. These configurable systems provide high throughput and rapid start up as well as a high degree of flexibility and customization. The manufacturers of such systems provide detailed protocols the various high throughput. Thus, for example, Zymark Corp. provides technical bulletins describing screening systems for detecting the modulation of gene transcription, ligand binding, and the like.

IV. Inhibition of MKP-1 Upregulation/Activity in Response to Oxidized Phospholipids.

It is demonstrated herein that inhibition of MKP-1 upregulation in response to oxidized phospholipids results in an inhibition/amelioration of symptoms associated with atherosclerosis. In particular aspects of the inflammatory response characteristic of plaque formation (e.g. monocyte recruitment and attachment to vascular endothelium) are mitigated. This results in mitigation of the consequences of plaque formation, e.g., myocardial infarction, stroke, vascular occlusion, hypertension, etc. In addition, the inflammatory response characteristic of rheumatoid arthritis rheumatoid arthritis lupus, viral infections, and/or osteoporosis is reduced/eliminated.

MKP-1 expression can be inhibited using a wide variety of approaches that include, but are not limited to antisense molecules, MKP-1 specific ribozymes, MKP-1 specific catalytic DNAs, intrabodies directed against MKP-1 proteins, lipids/phospholipids that competitively (or non-competitively) inhibit the effects of oxidized phospholipids, gene therapy approaches that knock out MKP-1, and small organic molecules that inhibit MKP-1 expression/Overexpression or block a receptor that is required to induce MKP-1.

A) Antisense Approaches.

MKP-1 gene regulation can be downregulated or entirely inhibited by the use of antisense molecules. An “antisense sequence or antisense nucleic acid” is a nucleic acid that is complementary to the coding MKP-1 mRNA nucleic acid sequence or a subsequence thereof. Binding of the antisense molecule to the MKP-1 mRNA interferes with normal translation of the MKP-1 polypeptide.

Thus, in accordance with preferred embodiments of this invention, preferred antisense molecules include oligonucleotides and oligonucleotide analogs that are hybridizable with MKP-1 messenger RNA. This relationship is commonly denominated as “antisense.” The oligonucleotides and oligonucleotide analogs are able to inhibit the function of the RNA, either its translation into protein, its translocation into the cytoplasm, or any other activity necessary to its overall biological function. The failure of the messenger RNA to perform all or part of its function results in a reduction or complete inhibition of expression of MKP-1 polypeptides.

In the context of this invention, the term “oligonucleotide” refers to a polynucleotide formed from naturally-occurring bases and/or cyclofuranosyl groups joined by native phosphodiester bonds. This term effectively refers to naturally-occurring species or synthetic species formed from naturally-occurring subunits or their close homologs. The term “oligonucleotide” may also refer to moieties which function similarly to oligonucleotides, but which have non naturally-occurring portions. Thus, oligonucleotides may have altered sugar moieties or inter-sugar linkages. Exemplary among these are the phosphorothioate and other sulfur containing species which are known for use in the art. In accordance with some preferred embodiments, at least one of the phosphodiester bonds of the oligonucleotide has been substituted with a structure which functions to enhance the ability of the compositions to penetrate into the region of cells where the RNA whose activity is to be modulated is located. It is preferred that such substitutions comprise phosphorothioate bonds, methyl phosphonate bonds, or short chain alkyl or cycloalkyl structures. In accordance with other preferred embodiments, the phosphodiester bonds are substituted with structures which are, at once, substantially non-ionic and non-chiral, or with structures which are chiral and enantiomerically specific. Persons of ordinary skill in the art will be able to select other linkages for use in the practice of the invention.

In one particularly preferred embodiment, the internucleotide phosphodiester linkage is replaced with a peptide linkage. Such peptide nucleic acids tend to show improved stability, penetrate the cell more easily, and show enhances affinity for their target. Methods of making peptide nucleic acids are known to those of skill in the art (see, e.g., U.S. Pat. Nos. 6,015,887, 6,015,710, 5,986,053, 5,977,296, 5,902,786, 5,864,010, 5,786,461, 5,773,571, 5,766,855, 5,736,336, 5,719,262, and 5,714,331).

Oligonucleotides may also include species which include at least some modified base forms. Thus, purines and pyrimidines other than those normally found in nature may be so employed. Similarly, modifications on the furanosyl portions of the nucleotide subunits may also be effected, as long as the essential tenets of this invention are adhered to. Examples of such modifications are 2′-O-alkyl- and 2′-halogen-substituted nucleotides. Some specific examples of modifications at the 2′ position of sugar moieties which are useful in the present invention are OH, SH, SCH₃, F, OCH₃, OCN, O(CH₂)[n]NH₂ or O(CH₂)[n]CH₃, where n is from 1 to about 10, and other substituents having similar properties.

Such oligonucleotides are best described as being functionally interchangeable with natural oligonucleotides or synthesized oligonucleotides along natural lines, but which have one or more differences from natural structure. All such analogs are comprehended by this invention so long as they function effectively to hybridize with messenger RNA of MKP-1 to inhibit the function of that RNA.

The oligonucleotides in accordance with this invention preferably comprise from about 3 to about 50 subunits. It is more preferred that such oligonucleotides and analogs comprise from about 8 to about 25 subunits and still more preferred to have from about 12 to about 20 subunits. As will be appreciated, a subunit is a base and sugar combination suitably bound to adjacent subunits through phosphodiester or other bonds. The oligonucleotides used in accordance with this invention may be conveniently and routinely made through the well-known technique of solid phase synthesis. Equipment for such synthesis is sold by several vendors, including Applied Biosystems. Any other means for such synthesis may also be employed, however, the actual synthesis of the oligonucleotides is well within the talents of the routineer. It is also will known to prepare other oligonucleotide such as phosphorothioates and alkylated derivatives.

Using the known sequence of the MKP-1 gene/cDNA, appropriate and effective antisense oligonucleotide sequences can be readily determined. One such sequence, used in Example 1, is 5′-GGA ACT CAG TGG AAC TCA GG-3′ (SEQ ID NO:2)

B) Catalytic RNAs and DNAs

1) Ribozymes.

In another approach, MKP-1 expression can be inhibited by the use of ribozymes. As used herein, “ribozymes” are include RNA molecules that contain anti-sense sequences for specific recognition, and an RNA-cleaving enzymatic activity. The catalytic strand cleaves a specific site in a target (MKP-1) RNA, preferably at greater than stoichiometric concentration. Two “types” of ribozymes are particularly useful in this invention, the hammerhead ribozyme (Rossi et al. (1991) Pharmac. Ther. 50: 245-254) and the hairpin ribozyme (Hampel et al. (1990) Nucl. Acids Res. 18: 299-304, and U.S. Pat. No. 5,254,678).

Because both hammerhead and hairpin ribozymes are catalytic molecules having antisense and endoribonucleotidase activity, ribozyme technology has emerged as a potentially powerful extension of the antisense approach to gene inactivation. The ribozymes of the invention typically consist of RNA, but such ribozymes may also be composed of nucleic acid molecules comprising chimeric nucleic acid sequences (such as DNA/RNA sequences) and/or nucleic acid analogs (e.g., phosphorothioates).

Accordingly, within one aspect of the present invention ribozymes are provided which have the ability to MKP-1 expression. Such ribozymes may be in the form of a “hammerhead” (for example, as described by Forster and Symons (1987) Cell 48: 211-220,; Haseloff and Gerlach (1988) Nature 328: 596-600; Walbot and Bruening (1988) Nature 334: 196; Haseloff and Gerlach (1988) Nature 334: 585) or a “hairpin” (see, e.g. U.S. Pat. No. 5,254,678 and Hampel et al., European Patent Publication No. 0 360 257, published Mar. 26, 1990), and have the ability to specifically target, cleave and MKP-1 nucleic acids.

The sequence requirement for the hairpin ribozyme is any RNA sequence consisting of NNNBN*GUCNNNNNN (where N*G is the cleavage site, where B is any of G, C, or U, and where N is any of G, U, C, or A) (SEQ ID NO: ______). Suitable MKP-1 of recognition or target sequences for hairpin ribozymes can be readily determined from the MKP-1 sequence. Certain appropriate sequences include, but are not limited to sequences used as targets for antisense molecules.

The sequence requirement at the cleavage site for the hammerhead ribozyme is any RNA sequence consisting of NUX (where N is any of G, U, C, or A and X represents C, U, or A) can be targeted. Accordingly, the same target within the hairpin leader sequence, GUC, is useful for the hammerhead ribozyme. The additional nucleotides of the hammerhead ribozyme or hairpin ribozyme is determined by the target flanking nucleotides and the hammerhead consensus sequence (see Ruffner et al. (1990) Biochemistry 29: 10695-10702).

Cech et al. (U.S. Pat. No. 4,987,071,) has disclosed the preparation and use of certain synthetic ribozymes which have endoribonuclease activity. These ribozymes are based on the properties of the Tetrahymena ribosomal RNA self-splicing reaction and require an eight base pair target site. A temperature optimum of 50° C. is reported for the endoribonuclease activity. The fragments that arise from cleavage contain 5′ phosphate and 3′ hydroxyl groups and a free guanosine nucleotide added to the 5′ end of the cleaved RNA. The preferred ribozymes of this invention hybridize efficiently to target sequences at physiological temperatures, making them particularly well suited for use in vivo

The ribozymes of this invention, as well as DNA encoding such ribozymes and other suitable nucleic acid molecules can be chemically synthesized using methods well known in the art for the synthesis of nucleic acid molecules. Alternatively, Promega, Madison, Wis., USA, provides a series of protocols suitable for the production of RNA molecules such as ribozymes. The ribozymes also can be prepared from a DNA molecule or other nucleic acid molecule (which, upon transcription, yields an RNA molecule) operably linked to an RNA polymerase promoter, e.g., the promoter for T7 RNA polymerase or SP6 RNA polymerase. Such a construct may be referred to as a vector. Accordingly, also provided by this invention are nucleic acid molecules, e.g., DNA or cDNA, coding for the ribozymes of this invention. When the vector also contains an RNA polymerase promoter operably linked to the DNA molecule, the ribozyme can be produced in vitro upon incubation with the RNA polymerase and appropriate nucleotides. In a separate embodiment, the DNA may be inserted into an expression cassette (see, e.g., Cotten and Birnstiel (1989) EMBO J 8(12):3861-3866; Hempel et al. (1989) Biochem. 28: 4929-4933, etc.).

After synthesis, the ribozyme can be modified by ligation to a DNA molecule having the ability to stabilize the ribozyme and make it resistant to RNase. Alternatively, the ribozyme can be modified to the phosphothio analog for use in liposome delivery systems. This modification also renders the ribozyme resistant to endonuclease activity.

The ribozyme molecule also can be in a host prokaryotic or eukaryotic cell in culture or in the cells of an organism/patient. Appropriate prokaryotic and eukaryotic cells can be transfected with an appropriate transfer vector containing the DNA molecule encoding a ribozyme of this invention. Alternatively, the ribozyme molecule, including nucleic acid molecules encoding the ribozyme, may be introduced into the host cell using traditional methods such as transformation using calcium phosphate precipitation (Dubensky et al. (1984) Proc. Natl. Acad. Sci., USA, 81: 7529-7533), direct microinjection of such nucleic acid molecules into intact target cells (Acsadi et al. (1991) Nature 352: 815-818), and electroporation whereby cells suspended in a conducting solution are subjected to an intense electric field in order to transiently polarize the membrane, allowing entry of the nucleic acid molecules. Other procedures include the use of nucleic acid molecules linked to an inactive adenovirus (Cotton et al. (1990) Proc. Natl. Acad. Sci., USA, 89 :6094), lipofection (Felgner et al. (1989) Proc. Natl. Acad. Sci. USA 84: 7413-7417), microprojectile bombardment (Williams et al. (1991) Proc. Natl. Acad. Sci., USA, 88: 2726-2730), polycation compounds such as polylysine, receptor specific ligands, liposomes entrapping the nucleic acid molecules, spheroplast fusion whereby E coli containing the nucleic acid molecules are stripped of their outer cell walls and fused to animal cells using polyethylene glycol, viral transduction, (Cline et al., (1985) Pharmac. Ther. 29: 69; and Friedmann et al. (1989) Science 244: 1275), and DNA ligand (Wu et al (1989) J. Biol. Chem. 264: 16985-16987), as well as psoralen inactivated viruses such as Sendai or Adenovirus. In one preferred embodiment, the ribozyme is introduced into the host cell utilizing a lipid, a liposome or a retroviral vector.

When the DNA molecule is operatively linked to a promoter for RNA transcription, the RNA can be produced in the host cell when the host cell is grown under suitable conditions favoring transcription of the DNA molecule. The vector can be, but is not limited to, a plasmid, a virus, a retrotransposon or a cosmid. Examples of such vectors are disclosed in U.S. Pat. No. 5,166,320. Other representative vectors include, but are not limited to adenoviral vectors (e.g., WO 94/26914, WO 93/9191; Kolls et al. (1994) PNAS 91(1):215-219; Kass-Eisler et al., (1993) Proc. Natl. Acad. Sci., USA, 90(24): 11498-502, Guzman et al. (1993) Circulation 88(6): 2838-48, 1993; Guzman et al. (1993) Cir. Res. 73(6):1202-1207, 1993; Zabner et al. (1993) Cell 75(2): 207-216; Li et al. (1993) Hum Gene Ther. 4(4): 403-409; Caillaud et al. (1993) Eur. J. Neurosci. 5(10): 1287-1291), adeno-associated vector type 1 (“AAV-1”) or adeno-associated vector type 2 (“AAV-2”) (see WO 95/13365; Flotte et al. (1993) Proc. Natl. Acad. Sci., USA, 90(22):10613-10617), retroviral vectors (e.g., EP 0 415 731; WO 90/07936; WO 91/02805; WO 94/03622; WO 93/25698; WO 93/25234; U.S. Pat. No. 5,219,740; WO 93/11230; WO 93/10218) and herpes viral vectors (e.g., U.S. Pat. No. 5,288,641). Methods of utilizing such vectors in gene therapy are well known in the art, see, for example, Larrick and Burck (1991) Gene Therapy: Application of Molecular Biology, Elsevier Science Publishing Co., Inc., New York, N.Y. , and Kreigler (1990) Gene Transfer and Expression: A Laboratory Manual, W. H. Freeman and Company, New York.

To produce ribozymes in vivo utilizing vectors, the nucleotide sequences coding for ribozymes are preferably placed under the control of a strong promoter such as the lac, SV40 late, SV40 early, or lambda promoters. Ribozymes are then produced directly from the transfer vector in vivo. Suitable transfector vectors for in vivo expression are discussed below.

2) Catalytic DNA

In a manner analogous to ribozymes, DNAs are also capable of demonstrating catalytic (e.g. nuclease) activity. While no such naturally-occurring DNAs are known, highly catalytic species have been developed by directed evolution and selection. Beginning with a population of 10¹⁴DNAs containing 50 random nucleotides, successive rounds of selective amplification, enriched for individuals that best promote the Pb²⁺-dependent cleavage of a target ribonucleoside 3′-O-P bond embedded within an otherwise all-DNA sequence. By the fifth round, the population as a whole carried out this reaction at a rate of 0.2 min⁻¹. Based on the sequence of 20 individuals isolated from this population, a simplified version of the catalytic domain that operates in an intermolecular context with a turnover rate of 1 min⁻¹ (see, e.g., Breaker and Joyce (1994) Chem Biol 4: 223-229.

In later work, using a similar strategy, a DNA enzyme was made that could cleave almost any targeted RNA substrate under simulated physiological conditions. The enzyme is comprised of a catalytic domain of 15 deoxynucleotides, flanked by two substrate-recognition domains of seven to eight deoxynucleotides each. The RNA substrate is bound through Watson-Crick base pairing and is cleaved at a particular phosphodiester located between an unpaired purine and a paired pyrimidine residue. Despite its small size, the DNA enzyme has a catalytic efficiency (kcat/Km) of approximately 10⁹ M⁻¹ min⁻¹ under multiple turnover conditions, exceeding that of any other known nucleic acid enzyme. By changing the sequence of the substrate-recognition domains, the DNA enzyme can be made to target different RNA substrates (Santoro and Joyce (1997) Proc. Natl. Acad. Sci., USA, 94(9): 4262-4266). Modifying the appropriate targeting sequences (e.g. as described by Santoro and Joyce, supra.) the DNA enzyme can easily be retargeted to MKP-1 mRNA thereby acting like a ribozyme.

C) Knocking out MKP-1

In another approach, MKP-1 can be inhibited/downregulated simply by “knocking out” the gene. Typically this is accomplished by disrupting the MKP-1 gene, the promoter regulating the gene or sequences between the promoter and the gene. Such disruption can be specifically directed to MKP-1 by homologous recombination where a “knockout construct” contains flanking sequences complementary to the domain to which the construct is targeted. Insertion of the knockout construct (e.g. into the MKP-1 gene) results in disruption of that gene. The phrases “disruption of the gene” and “gene disruption” refer to insertion of a nucleic acid sequence into one region of the native DNA sequence (usually one or more exons) and/or the promoter region of a gene so as to decrease or prevent expression of that gene in the cell as compared to the wild-type or naturally occurring sequence of the gene. By way of example, a nucleic acid construct can be prepared containing a DNA sequence encoding an antibiotic resistance gene which is inserted into the DNA sequence that is complementary to the DNA sequence (promoter and/or coding region) to be disrupted. When this nucleic acid construct is then transfected into a cell, the construct will integrate into the genomic DNA. Thus, the cell and its progeny will no longer express the gene or will express it at a decreased level, as the DNA is now disrupted by the antibiotic resistance gene.

Knockout constructs can be produced by standard methods known to those of skill in the art. The knockout construct can be chemically synthesized or assembled, e.g., using recombinant DNA methods. The DNA sequence to be used in producing the knockout construct is digested with a particular restriction enzyme selected to cut at a location(s) such that a new DNA sequence encoding a marker gene can be inserted in the proper position within this DNA sequence. The proper position for marker gene insertion is that which will serve to prevent expression of the native gene; this position will depend on various factors such as the restriction sites in the sequence to be cut, and whether an exon sequence or a promoter sequence, or both is (are) to be interrupted (i.e., the precise location of insertion necessary to inhibit promoter function or to inhibit synthesis of the native exon). Preferably, the enzyme selected for cutting the DNA will generate a longer arm and a shorter arm, where the shorter arm is at least about 300 base pairs (bp). In some cases, it will be desirable to actually remove a portion or even all of one or more exons of the gene to be suppressed so as to keep the length of the knockout construct comparable to the original genomic sequence when the marker gene is inserted in the knockout construct. In these cases, the genomic DNA is cut with appropriate restriction endonucleases such that a fragment of the proper size can be removed.

The marker gene can be any nucleic acid sequence that is detectable and/or assayable, however typically it is an antibiotic resistance gene or other gene whose expression or presence in the genome can easily be detected. The marker gene is usually operably linked to its own promoter or to another strong promoter from any source that will be active or can easily be activated in the cell into which it is inserted; however, the marker gene need not have its own promoter attached as it may be transcribed using the promoter of the gene to be suppressed. In addition, the marker gene will normally have a polyA sequence attached to the 3′ end of the gene; this sequence serves to terminate transcription of the gene. Preferred marker genes are any antibiotic resistance gene including, but not limited to neo (the neomycin resistance gene) and beta-gal (beta-galactosidase).

After the genomic DNA sequence has been digested with the appropriate restriction enzymes, the marker gene sequence is ligated into the genomic DNA sequence using methods well known to the skilled artisan (see, e.g., Berger and Kimmel, Guide to Molecular Cloning Techniques, Methods in Enzymology volume 152 Academic Press, Inc., San Diego, Calif.; Sambrook et al. (1989) Molecular Cloning—A Laboratory Manual (2nd ed.) Vol. 1-3, Cold Spring Harbor Laboratory, Cold Spring Harbor Press, NY; and Current Protocols in Molecular Biology, F. M. Ausubel et al., eds., Current Protocols, a joint venture between Greene Publishing Associates, Inc. and John Wiley & Sons, Inc., (1994) Supplement). The ends of the DNA fragments to be ligated must be compatible; this is achieved by either cutting all fragments with enzymes that generate compatible ends, or by blunting the ends prior to ligation. Blunting is done using methods well known in the art, such as for example by the use of Klenow fragment (DNA polymerase I) to fill in sticky ends.

Suitable knockout constructs have been made and used to produce MKP-1 knockout mice (see, e.g., Dorfman et al. (1996) Oncogene 13: 925-931). The knockout constructs can be delivered to cells in vivo using gene therapy delivery vehicles (e.g. retroviruses, liposomes, lipids, dendrimers, etc.) as described below. Methods of knocking out genes are well described in the literature and essentially routine to those of skill in the art (see, e.g., Thomas et al. (1986) Cell 44(3): 419-428; Thomas, et al. (1987) Cell 51(3): 503-512)1; Jasin and Berg (1988) Genes & Development 2: 1353-1363; Mansour, et al. (1998) Nature 336: 348-352; Brinster, et al. (1989) Proc Natl Acad Sci 86: 7087-7091; Capecchi (1989) Trends in Genetics 5(3): 70-76; Frohman and Martin (1989) Cell 56: 145-147; Hasty, et al. (1991) Mol Cell Bio 11(11): 5586-5591; Jeannotte, et al. (1991) Mol Cell Biol. 11(11): 557814 5585; and Mortensen, et al. (1992) Mol Cell Biol. 12(5): 2391-2395

The use of homologous recombination to alter expression of endogenous genes is also described in detail in U.S. Pat. No. 5,272,071, WO 91/09955, WO 93/09222, WO 96/29411, WO 95/31560, and WO 91/12650.

D) Intrabodies.

In still another embodiment, MKP-1 expression/activity is inhibited by transfecting the subject cell(s) (e.g., cells of the vascular endothelium) with a nucleic acid construct that expresses an intrabody. An intrabody is an intracellular antibody, in this case, capable of recognizing and binding to a MKP-1 polypeptide. The intrabody is expressed by an “antibody cassette”, containing a sufficient number of nucleotides coding for the portion of an antibody capable of binding to the target (MKP-1 polypeptide) operably linked to a promoter that will permit expression of the antibody in the cell(s) of interest. The construct encoding the intrabody is delivered to the cell where the antibody is expressed intracellularly and binds to the target MKP-1, thereby disrupting the target from its normal action. This antibody is sometimes referred to as an “intrabody”.

In one preferred embodiment, the “intrabody gene” (antibody) of the antibody cassette would utilize a cDNA, encoding heavy chain variable (V_{H}) and light chain variable (V_{L}) domains of an antibody which can be connected at the DNA level by an appropriate oligonucleotide as a bridge of the two variable domains, which on translation, form a single peptide (referred to as a single chain variable fragment, “sFv”) capable of binding to a target such as an MKP-1 protein. The intrabody gene preferably does not encode an operable secretory sequence and thus the expressed antibody remains within the cell.

Anti-MKP-1 antibodies suitable for use/expression as intrabodies in the methods of this invention can be readily produced by a variety of methods. Such methods include, but are not limited to, traditional methods of raising “whole” polyclonal antibodies, which can be modified to form single chain antibodies, or screening of, e.g. phage display libraries to select for antibodies showing high specificity and/or avidity for MKP-1. Such screening methods are described above in some detail.

The antibody cassette is delivered to the cell by any of the known means. One preferred delivery system is described in U.S. patent application Ser. No. 08/199,070 by Marasco filed Feb. 22, 1994, which is incorporated herein by reference. This discloses the use of a fusion protein comprising a target moiety and a binding moiety. The target moiety brings the vector to the cell, while the binding moiety carries the antibody cassette. Other methods include, for example, Miller (1992) Nature 357: 455-460; Anderson (1992) Science 256: 808-813; Wu, et al. (1988) J. Biol. Chem. 263: 14621-14624. For example, a cassette containing these (anti-MKP-1) antibody genes, such as the sFv gene, can be targeted to a particular cell by a number of techniques including, but not limited to the use of tissue-specific promoters, the use of tissue specific vectors, and the like. Methods of making and using intrabodies are described in detail in U.S. Pat. No. 6,004,940.

E) Blocking Access of Oxidized Phospholipids to Target Signal Transduction Molecules.

As indicated above, oxidized phospholipids induce a strong upregulation of MKP-1. Without being bound to a particular theory, it is believed this response is mediated by the interaction of the oxidized phospholipid with a receptor and/or with one or more signaling proteins. By blocking access of the oxidized phospholipid to the target signal transduction molecule, upregulation of MKP-1 in response to oxidized lipids can be accomplished.

One approach to such blocking is the use of competitive inhibitors. In one preferred embodiment such competitive inhibitors include lipids (preferably non-oxidized) or other hydrophobic and/or amphipathic molecules. The inhibitors (competitive or non-competitive) bind to, or interact with, the receptor/signaling protein thereby rendering it unavailable to oxidized phospholipids (or other relevant fractions of LDL). The lipid/hydrophobic molecule can be delivered to the target site by any of a number of methods well known to those of skill in the art (e.g. using small organic molecules or targeted liposomes, tissue-specific lipids, etc.).

Suitable lipid, hydrophobic, or amphipathic molecules can be identified with only routine screening using the screening methods of this invention.

F) Small Organic Molecules.

In still another embodiment, MKP-1 expression and/or MKP-1 protein activity can be inhibited by the use of small organic molecules. Such molecules include, but are not limited to molecules that specifically bind to the DNA comprising the MKP-1 promoter and/or coding region, molecules that bind to and complex with MKP-1 mRNA, molecules that inhibit the signaling pathway that results in MKP-1 upregulation, and molecules that bind to and/or compete with MKP-1 polypeptides. Small organic molecules effective at inhibiting MKP-1 expression can be identified with routine screening using the methods described herein.

The methods of inhibiting MKP-1 expression described above are meant to be illustrative and not limiting. In view of the teachings provided herein, other methods of inhibiting MKP-1 will be known to those of skill in the art.

G) Modes of Administration.

The mode of administration of the MKP-1 blocking agent depends on the nature of the particular agent. Antisense molecules, catalytic RNAs (ribozymes), catalytic DNAs, small organic molecules, and other molecules (e.g. lipids, antibodies, etc.) used as MKP-1 inhibitors may be formulated as pharmaceuticals (e.g. with suitable excipient) and delivered using standard pharmaceutical formulation and delivery methods as described below. Antisense molecules, catalytic RNAs (ribozymes), catalytic DNAs, and additionally, knockout constructs, and constructs encoding intrabodies can be delivered and (if necessary) expressed in target cells (e.g. vascular endothelial cells) using methods of gene therapy, e.g. as described below.

1) Pharmaceutical Administration.

In order to carry out the methods of the invention, lipids and/or one or more inhibitors of MKP-1 expression (e.g. ribozymes, antibodies, antisense molecules, small organic molecules, etc.) are administered to an individual to ameliorate one or more symptoms of atherosclerosis, lupus, viral infectons, osteoporosis, and/or rheumatoid arthritis. While this invention is described generally with reference to human subjects, veterinary applications are contemplated within the scope of this invention.

Various inhibitors may be administered, if desired, in the form of salts, esters, amides, prodrugs, derivatives, and the like, provided the salt, ester, amide, prodrug or derivative is suitable pharmacologically, i.e., effective in the present method. Salts, esters, amides, prodrugs and other derivatives of the active agents may be prepared using standard procedures known to those skilled in the art of synthetic organic chemistry and described, for example, by March (1992) Advanced Organic Chemistry; Reactions, Mechanisms and Structure, 4th Ed. N.Y. Wiley-Interscience.

The lipids and/or MKP-1 inhibitors and various derivatives and/or formulations thereof are useful for parenteral, topical, oral, or local administration, such as by aerosol or transdermally, for prophylactic and/or therapeutic treatment of coronary disease and/or rheumatoid arthritis. The pharmaceutical compositions can be administered in a variety of unit dose forms depending upon the method of administration. Suitable unit dose forms, include, but are not limited to powders, tablets, pills, capsules, lozenges, suppositories, etc.

The lipids and/or MKP-1 inhibitors and various derivatives and/or formulations thereof are typically combined with a pharmaceutically acceptable carrier (excipient) to form a pharmacological composition. Pharmaceutically acceptable carriers can contain one or more physiologically acceptable compound(s) that act, for example, to stabilize the composition or to increase or decrease the absorption of the active agent(s). Physiologically acceptable compounds can include, for example, carbohydrates, such as glucose, sucrose, or dextrans, antioxidants, such as ascorbic acid or glutathione, chelating agents, low molecular weight proteins, compositions that reduce the clearance or hydrolysis of the active agents, or excipients or other stabilizers and/or buffers.

Other physiologically acceptable compounds include wetting agents, emulsifying agents, dispersing agents or preservatives which are particularly useful for preventing the growth or action of microorganisms. Various preservatives are well known and include, for example, phenol and ascorbic acid. One skilled in the art would appreciate that the choice of pharmaceutically acceptable carrier(s), including a physiologically acceptable compound depends, for example, on the route of administration of the active agent(s) and on the particular physio-chemical characteristics of the active agent(s). The excipients are preferably sterile and generally free of undesirable matter. These compositions may be sterilized by conventional, well-known sterilization techniques.

The concentration of active agent(s) in the formulation can vary widely, and will be selected primarily based on fluid volumes, viscosities, body weight and the like in accordance with the particular mode of administration selected and the patient's needs.

In therapeutic applications, the compositions of this invention are administered to a patient suffering from a disease (e.g., atherosclerosis and/or associated conditions, and/or rheumatoid arthritis) in an amount sufficient to cure or at least partially arrest the disease and/or its symptoms (e.g. to reduce plaque formation, to reduce monocyte recruitment, etc.) An amount adequate to accomplish this is defined as a “therapeutically effective dose.” Amounts effective for this use will depend upon the severity of the disease and the general state of the patient's health. Single or multiple administrations of the compositions may be administered depending on the dosage and frequency as required and tolerated by the patient. In any event, the composition should provide a sufficient quantity of the active agents of the formulations of this invention to effectively treat (ameliorate one or more symptoms) the patient.

In certain preferred embodiments, the lipids and/or MKP-1 inhibitors are administered orally (e.g. via a tablet) or as an injectable in accordance with standard methods well known to those of skill in the art. In other preferred embodiments, the MKP-1 inhibitors may also be delivered through the skin using conventional transdermal drug delivery systems, i.e., transdermal “patches” wherein the active agent(s) are typically contained within a laminated structure that serves as a drug delivery device to be affixed to the skin. In such a structure, the drug composition is typically contained in a layer, or “reservoir,” underlying an upper backing layer. It will be appreciated that the term “reservoir” in this context refers to a quantity of “active ingredient(s)” that is ultimately available for delivery to the surface of the skin. Thus, for example, the “reservoir” may include the active ingredient(s) in an adhesive on a backing layer of the patch, or in any of a variety of different matrix formulations known to those of skill in the art. The patch may contain a single reservoir, or it may contain multiple reservoirs.

In one embodiment, the reservoir comprises a polymeric matrix of a pharmaceutically acceptable contact adhesive material that serves to affix the system to the skin during drug delivery. Examples of suitable skin contact adhesive materials include, but are not limited to, polyethylenes, polysiloxanes, polyisobutylenes, polyacrylates, polyurethanes, and the like. Alternatively, the drug-containing reservoir and skin contact adhesive are present as separate and distinct layers, with the adhesive underlying the reservoir which, in this case, may be either a polymeric matrix as described above, or it may be a liquid or hydrogel reservoir, or may take some other form. The backing layer in these laminates, which serves as the upper surface of the device, preferably functions as a primary structural element of the “patch” and provides the device with much of its flexibility. The material selected for the backing layer is preferably substantially impermeable to the active agent(s) and any other materials that are present.

The foregoing formulations and administration methods are intended to be illustrative and not limiting. It will be appreciated that, using the teaching provided herein, other suitable formulations and modes of administration can be readily devised.

2) Gene Therapy.

As indicated above, antisense molecules, catalytic RNAs (ribozymes), catalytic DNAs, and additionally, knockout constructs, and constructs encoding intrabodies can be delivered and transcribed and/or expressed in target cells (e.g. vascular endothelial cells) using methods of gene therapy. Thus, in certain preferred embodiments, the nucleic acids encoding knockout constructs, intrabodies, antisense molecules, catalytic RNAs or DNAs, etc. are cloned into gene therapy vectors that are competent to transfect cells (such as human or other mammalian cells) in vitro and/or in vivo.

Many approaches for introducing nucleic acids into cells in vivo, ex vivo and in vitro are known. These include lipid or liposome based gene delivery (WO 96/18372; WO 93/24640; Mannino and Gould-Fogerite (1988) BioTechniques 6(7): 682-691; Rose U.S. Pat. No. 5,279,833; WO 91/06309; and Felgner et al. (1987) Proc. Natl. Acad. Sci. USA 84: 7413-7414) and replication-defective retroviral vectors harboring a therapeutic polynucleotide sequence as part of the retroviral genome (see, e.g., Miller et al. (1990) Mol. Cell. Biol. 10:4239 (1990); Kolberg (1992) J. NIH Res. 4: 43, and Cornetta et al. (1991) Hum. Gene Ther. 2: 215).

For a review of gene therapy procedures, see, e.g., Anderson, Science (1992) 256: 808-813; Nabel and Felgner (1993) TIBTECH 11: 211-217; Mitani and Caskey (1993) TIBTECH 11: 162-166; Mulligan (1993) Science, 926-932; Dillon (1993) TIBTECH 11: 167-175; Miller (1992) Nature 357: 455-460; Van Brunt (1988) Biotechnology 6(10): 1149-1154; Vigne (1995) Restorative Neurology and Neuroscience 8: 35-36; Kremer and Perricaudet (1995) British Medical Bulletin 51(1) 31-44; Haddada et al. (1995) in Current Topics in Microbiology and Immunology, Doerfler and Böhm (eds) Springer-Verlag, Heidelberg Germany; and Yu et al., (1994) Gene Therapy, 1:13-26.

Widely used retroviral vectors include those based upon murine leukemia virus (MuLV), gibbon ape leukemia virus (GaLV), Simian Immunodeficiency virus (SIV), human immunodeficiency virus (HIV), alphavirus, and combinations thereof (see, e.g., Buchscher et al. (1992) J. Virol. 66(5) 2731-2739; Johann et al. (1992) J. Virol. 66 (5):1635-1640 (1992); Sommerfelt et al., (1990) Virol. 176:58-59; Wilson et al. (1989) J. Virol. 63:2374-2378; Miller et al., J. Virol. 65:2220-2224 (1991); Wong-Staal et al., PCT/US94/05700, and Rosenburg and Fauci (1993) in Fundamental Immunology, Third Edition Paul (ed) Raven Press, Ltd., New York and the references therein, and Yu et al. (1994) Gene Therapy, supra; U.S. Pat. No. 6,008,535, and the like).

The vectors are optionally pseudotyped to extend the host range of the vector to cells which are not infected by the retrovirus corresponding to the vector. For example, the vesicular stomatitis virus envelope glycoprotein (VSV-G) has been used to construct VSV-G-pseudotyped HIV vectors which can infect hematopoietic stem cells (Naldini et al. (1996) Science 272:263, and Akkina et al. (1996) J Virol 70:2581).

Adeno-associated virus (AAV)-based vectors are also used to transduce cells with target nucleic acids, e.g., in the in vitro production of nucleic acids and peptides, and in in vivo and ex vivo gene therapy procedures. See, West et al. (1987) Virology 160:38-47; Carter et al. (1989) U.S. Pat. No. 4,797,368; Carter et al. WO 93/24641 (1993); Kotin (1994) Human Gene Therapy 5:793-801; Muzyczka (1994) J. Clin. Invst. 94:1351 for an overview of AAV vectors. Construction of recombinant AAV vectors are described in a number of publications, including Lebkowski, U.S. Pat. No. 5,173,414; Tratschin et al. (1985) Mol. Cell. Biol. 5(11):3251-3260; Tratschin, et al. (1984) Mol. Cell. Biol., 4: 2072-2081; Hermonat and Muzyczka (1984) Proc. Natl. Acad. Sci. USA, 81: 6466-6470; McLaughlin et al. (1988) and Samulski et al. (1989) J. Virol., 63:03822-3828. Cell lines that can be transformed by rAAV include those described in Lebkowski et al. (1988) Mol. Cell. Biol., 8:3988-3996. Other suitable viral vectors include herpes virus, lentivirus, and vaccinia virus.

a) Retroviral Transfection Systems.

In one particularly preferred embodiment, retroviruses (e.g. lentiviruses) are used to transfect the target cell(s) with constructs that block or inhibit MKP-1 expression. Retroviruses, in particular lentiviruses (e.g. HIV, SIV, etc.) are particularly well suited for this application because they are capable of infecting a non-dividing cell. Methods of using retroviruses for nucleic acid transfection are known to those of skill in the art (see, e.g., U.S. Pat. No. 6,013, 576).

Retroviruses are RNA viruses wherein the viral genome is RNA. When a host cell is infected with a retrovirus, the genomic RNA is reverse transcribed into a DNA intermediate which is integrated very efficiently into the chromosomal DNA of infected cells. This integrated DNA intermediate is referred to as a provirus. Transcription of the provirus and assembly into infectious virus occurs in the presence of an appropriate helper virus or in a cell line containing appropriate sequences enabling encapsidation without coincident production of a contaminating helper virus. In preferred embodiments, a helper virus need not be utilized for the production of the recombinant retrovirus since the sequences for encapsidation can be provided by co-transfection with appropriate vectors.

The retroviral genome and the proviral DNA have three genes: the gag, the pol, and the env, which are flanked by two long terminal repeat (LTR) sequences. The gag gene encodes the internal structural (matrix, capsid, and nucleocapsid) proteins; the pol gene encodes the RNA-directed DNA polymerase (reverse transcriptase) and the env gene encodes viral envelope glycoproteins. The 5′ and 3′ LTRs serve to promote transcription and polyadenylation of the virion RNAs. The LTR contains all other cis-acting sequences necessary for viral replication. Lentiviruses have additional genes including vit, vpr, tat, rev, vpu, nef, and vpx (in HIV-1, HIV-2 and/or SIV).

Adjacent to the 5′ LTR are sequences necessary for reverse transcription of the genome (the tRNA primer binding site) and for efficient encapsidation of viral RNA into particles (the Psi site). If the sequences necessary for encapsidation (or packaging of retroviral RNA into infectious virions) are missing from the viral genome, the result is a cis defect which prevents encapsidation of genomic RNA. However, the resulting mutant is still capable of directing the synthesis of all virion proteins.

In one preferred embodiment, the invention provides a recombinant retrovirus capable of infecting a non-dividing cell. The recombinant retrovirus comprises a viral GAG, a viral POL, a viral ENV, a heterologous nucleic acid sequence operably linked to a regulatory nucleic acid sequence, and cis-acting nucleic acid sequences necessary for packaging, reverse transcription and integration, as described above. It should be understood that the recombinant retrovirus of the invention is capable of infecting dividing cells as well as non-dividing cells.

In preferred embodiments, the recombinant retrovirus is therefore genetically modified in such a way that some of the structural, infectious genes of the native virus (e.g. env, gag, pol) have been removed and replaced instead with a nucleic acid sequence to be delivered to a target non-dividing cell (e.g., a sequence encoding the constructs that block or inhibit MKP-1 expression). After infection of a cell by the virus, the virus injects its nucleic acid into the cell and the retrovirus genetic material can integrate into the host cell genome. The transferred retrovirus genetic material is then transcribed and optionally translated within the host cell. Methods of making and using lentiviral vectors are discussed in detail in U.S. Pat. No. 6,013,516, 5,932,467, and the like.

2) Adenoviral Vector Systems.

In another preferred embodiment, the constructs that block or inhibit MKP-1 expression are expressed in an adenoviral vector suitable for gene therapy. The use of adenoviral vectors is described in detail in WO 96/25507. Particularly preferred adenoviral vectors are described by Wills et al. (1994) Hum. Gene Therap. 5: 1079-1088. Typically, adenoviral vectors contain a deletion in the adenovirus early region 3 and/or early region 4 and this deletion may include a deletion of some or all of the protein IX gene. In one embodiment, the adenoviral vectors include deletions of the E1a and/or E1b sequences.

A number of different adenoviral vectors have been optimized for gene transfer. One such adenoviral vector is described in U.S. Pat. No. 6,020,191. This vector comprises a CMV promoter to which a transgene may be operably linked and further contains an El deletion and a partial deletion of 1.6 kb from the E3 region. This is a replication defective vector containing a deletion in the El region into which a transgene (e.g. the β subunit gene) and its expression control sequences can be inserted, preferably the CMV promoter contained in this vector. It further contains the wild-type adenovirus E2 and E4 regions. The vector contains a deletion in the E3 region which encompasses 1549 nucleotides from adenovirus nucleotides 29292 to 30840 (Roberts et al. (1986) Adenovirus DNA, in Developments in Molecular Virology, W. Doerfler, ed., 8: 1-51). These modifications to the E3 region in the vector result in the following: (a) all the downstream splice acceptor sites in the E3 region are deleted and only mRNA a would be synthesized from the E3 promoter (Tollefson et al. (1996) J, Virol. 70:2 296-2306, 1996; Tollefson et al. (1996) Virology 220: 152-162,); (b) the E3A poly A site has been deleted, but the E3B poly A site has been retained; (c) the E3 gp19K (MHC I binding protein) gene has been retained; and (d) the E3 11.6K (Ad death protein) gene has been deleted.

Such adenoviral vectors can utilize adenovirus genomic sequences from any adenovirus serotypes, including but not limited to, adenovirus serotypes 2,5, and all other preferably non-oncogenic serotypes.

In one preferred embodiment, the cytomegalovirus (CMV) immediate early promoter (Boshart et al. (1985) Cell 41: 521-530) is used to control transcription and/or translation of constructs that block or inhibit MKP-1 expression, or a truncated fragment of this promoter which functions analogously may be used. The CMV promoter is positioned 5′ to the transgene(s) (e.g. constructs that block or inhibit MKP-1 expression) in a transcription unit. Portions of the full-length promoter can be tested for their ability to allow persistent expression of the transgene.

Polyadenylation signals which may be positioned at the 3′ end of the transgene in a include, but are not limited to, those derived from bovine growth hormone (BGH) and SV40.

To create the recombinant adenoviral vectors of the invention which contain a transcription unit (expression cassette) encoding a constructs that block or inhibit MKP-1 expression, a plasmid containing the transcription unit inserted into an adenovirus genomic fragment is co-transfected with a linearized viral genome derived from an adenoviral vector of interest into a recipient cell under conditions whereby homologous recombination occurs between the genomic fragment and the virus. Preferably, the transcription unit is engineered into the site of an El deletion. As a result, the transcription unit encoding a desired transgene is inserted into the adenoviral genome at the site in which it was cloned into the plasmid, creating a recombinant adenoviral vector. Following the homologous recombination, the vector genome is encapsidated into virions as evidenced by the formation of viral plaques. Preparation of replication-defective vector stocks can be accomplished using cell lines that complement viral genes deleted from the vector, e.g., 293 or A549 cells containing the deleted adenovirus El genomic sequences. After amplification of plaques in suitable complementing cell lines, the viruses can be recovered by freeze-thawing and subsequently purified using cesium chloride centrifugation. Alternatively, virus purification can be performed using chromatographic techniques (e.g., as set forth in International Application No. PCT/US96/13872.

Titers of replication-defective adenoviral vector stocks can be determined by plaque formation in a complementing cell line, e.g., 293 cells. For example, end-point dilution using an antibody to the adenoviral hexon protein may be used to quantitate virus production (Armentano et al. (195) Hum. Gene Ther. 6:1343-1353).

3) Non-Viral Transfection.

Alone, or in combination with viral vectors, a number of non-viral vectors are also useful for transfecting cells to express constructs that block or inhibit MKP-1 expression. Suitable non-viral vectors include, but are not limited to, plasmids, cosmids, phagemids, liposomes, water-oil emulsions, polethylene imines, biolistic pellets/beads, and dendrimers.

Liposomes were first described in 1965 as a model of cellular membranes and quickly were applied to the delivery of substances to cells. liposomes entrap DNA by one of two mechanisms which has resulted in their classification as either cationic liposomes or pH-sensitive liposomes. Cationic liposomes are positively charged liposomes which interact with the negatively charged DNA molecules to form a stable complex. Cationic liposomes typically consist of a positively charged lipid and a co-lipid. Commonly used co-lipids include dioleoyl phosphatidylethanolamine (DOPE) or dioleoyl phosphatidylcholine (DOPC). Co-lipids, also called helper lipids, are in most cases required for stabilization of liposome complex. A variety of positively charged lipid formulations are commercially available and many other are under development. Two of the most frequently cited cationic lipids are lipofectamine and lipofectin. Lipofectin is a commercially available cationic lipid first reported by Phil Felgner in 1987 to deliver genes to cells in culture. Lipofectin is a mixture of N-[1-(2,3-dioleyloyx) propyl]-N-N-N-trimethyl ammonia chloride (DOTMA) and DOPE.

DNA and lipofectin or lipofectamine interact spontaneously to form complexes that have a 100% loading efficiency. In other words, essentially all of the DNA is complexed with the lipid, provided enough lipid is available. It is assumed that the negative charge of the DNA molecule interacts with the positively charged groups of the DOTMA. The lipid:DNA ratio and overall lipid concentrations used in forming these complexes are extremely important for efficient gene transfer and vary with application. Lipofectin has been used to deliver linear DNA, plasmid DNA, and RNA to a variety of cells in culture. Shortly after its introduction, it was shown that lipofectin could be used to deliver genes in vivo. Following intravenous administration of lipofectin-DNA complexes, both the lung and liver showed marked affinity for uptake of these complexes and transgene expression. Injection of these complexes into other tissues has had varying results and, for the most part, are much less efficient than lipofectin-mediated gene transfer into either the lung or the liver.

PH-sensitive, or negatively-charged liposomes, entrap DNA rather than complex with it. Since both the DNA and the lipid are similarly charged, repulsion rather than complex formation occurs. Yet, some DNA does manage to get entrapped within the aqueous interior of these liposomes. In some cases, these liposomes are destabilized by low pH and hence the term pH-sensitive. To date, cationic liposomes have been much more efficient at gene delivery both in vivo and in vitro than pH-sensitive liposomes. pH-sensitive liposomes have the potential to be much more efficient at in vivo DNA delivery than their cationic counterparts and should be able to do so with reduced toxicity and interference from serum protein.

In another approach dendrimers complexed to the DNA have been used to transfect cells. Such dendrimers include, but are not limited to, “starburst” dendrimers and various dendrimer polycations.

Dendrimer polycations are three dimensional, highly ordered oligomeric and/or polymeric compounds typically formed on a core molecule or designated initiator by reiterative reaction sequences adding the oligomers and/or polymers and providing an outer surface that is positively changed. These dendrimers may be prepared as disclosed in PCT/US83/02052, and U.S. Pat. Nos. 4,507,466, 4,558,120, 4,568,737, 4,587,329, 4,631,337, 4,694,064, 4,713,975, 4,737,550, 4,871,779, 4,857,599.

Typically, the dendrimer polycations comprise a core molecule upon which polymers are added. The polymers may be oligomers or polymers which comprise terminal groups capable of acquiring a positive charge. Suitable core molecules comprise at least two reactive residues which can be utilized for the binding of the core molecule to the oligomers and/or polymers. Examples of the reactive residues are hydroxyl, ester, amino, imino, imido, halide, carboxyl, carboxyhalide maleimide, dithiopyridyl, and sulfhydryl, among others. Preferred core molecules are ammonia, tris-(2-aminoethyl)amine, lysine, omithine, pentaerythritol and ethylenediamine, among others. Combinations of these residues are also suitable as are other reactive residues.

Oligomers and polymers suitable for the preparation of the dendrimer polycations of the invention are pharmaceutically-acceptable oligomers and/or polymers that are well accepted in the body. Examples of these are polyamidoamines derived from the reaction of an alkyl ester of an α,β-ethylenically unsaturated carboxylic acid or an α,β-ethylenically unsaturated amide and an alkylene polyamine or a polyalkylene polyamine, among others. Preferred are methyl acrylate and ethylenediamine. The polymer is preferably covalently bound to the core molecule.

The terminal groups that may be attached to the oligomers and/or polymers should be capable of acquiring a positive charge. Examples of these are azoles and primary, secondary, tertiary and quaternary aliphatic and aromatic amines and azoles, which may be substituted with S or O, guanidinium, and combinations thereof. The terminal cationic groups are preferably attached in a covalent manner to the oligomers and/or polymers. Preferred terminal cationic groups are amines and guanidinium. However, others may also be utilized. The terminal cationic groups may be present in a proportion of about 10 to 100% of all terminal groups of the oligomer and/or polymer, and more preferably about 50 to 100%.

The dendrimer polycation may also comprise 0 to about 90% terminal reactive residues other than the cationic groups. Suitable terminal reactive residues other than the terminal cationic groups are hydroxyl, cyano, carboxyl, sulfhydryl, amide and thioether, among others, and combinations thereof. However others may also be utilized.

The dendrimer polycation is generally and preferably non-covalently associated with the polynucleotide. This permits an easy disassociation or disassembling of the composition once it is delivered into the cell. Typical dendrimer polycation suitable for use herein have a molecular weight ranging from about 2,000 to 1,000,000 Da, and more preferably about 5,000 to 500,000 Da. However, other molecule weights are also suitable. Preferred dendrimer polycations have a hydrodynamic radius of about 11 to 60 A., and more preferably about 15 to 55 A. Other sizes, however, are also suitable. Methods for the preparation and use of dendrimers in gene therapy are well known to those of skill in the art and describe in detail, for example, in U.S. Pat. No. 5,661,025

Where appropriate, two or more types of vectors can be used together. For example, a plasmid vector may be used in conjunction with liposomes. In the case of non-viral vectors, nucleic acid may be incorporated into the non-viral vectors by any suitable means known in the art. For plasmids, this typically involves ligating the construct into a suitable restriction site. For vectors such as liposomes, water-oil emulsions, polyethylene amines and dendrimers, the vector and construct may be associated by mixing under suitable conditions known in the art.

V. Use of Lipids to Amelioriate One or More Symptoms of an Inflammatory Response and/or Associated Pathologies.

In certain embodiments, this invention provides methods for mitigating one or more symptoms of atherosclerosis or other conditions characterized by an inflammatory response (e.g. rheumatoid arthritis, lupus erythematous, polyarteritis nodosa, osteoporosis, and viral illnesses such as influenza A, etc.). In particularly preferred embodiments, the invention pertains to the use of phospholipids to prevent LDL oxidation by artery cell walls and/or to inhibit other inflammatory processes. The methods involve administering a lipid, preferably a phospholipid to the organism (e.g. a human, a non-human mammal, etc.) in need of such treatment.

In our original observations we noted that high concentrations of phospholipid vesicles were capable of preventing LDL oxidation by artery wall cells. Monocyte transmigration induced by modification of low density lipoprotein in coculures of human aortic wall cells is due to induction of monocyte chemotactic protein 1 synthesis and is abolished by high density lipoproteins.

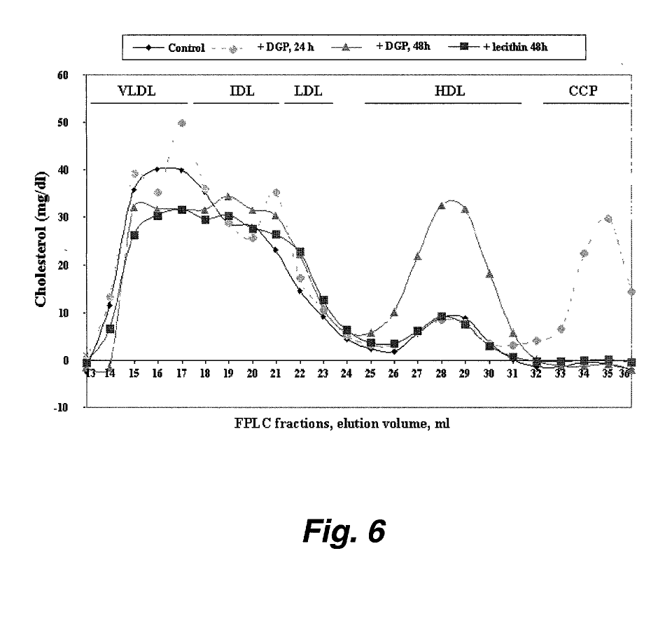

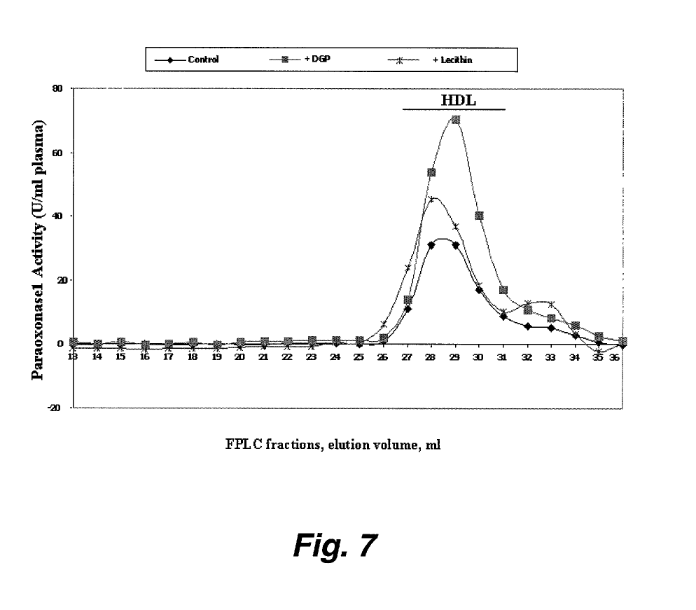

Without being bound to a particular theory, we believe the phospholipids (e.g. phospholipid vesicles) act as a sink that partitioned seeding molecules necessary for LDL oxidation. It is noted that the synthetic lipid 1,2-dimyristoyl-sn-glycero-3-phosphocholine (1,2-ditetradecanoyl-sn-glycero-3-phosphocholine) was particularly effective in raising HDL and paraoxonase levels and hence in making HDL much more anti-inflammatory (see, e.g., FIGS. 6 and 7).

Since many inflammatory conditions have been suspected to be mediated at least in part by oxidized lipids, we believe that this invention is also effective in ameliorating conditions that are known or suspected to be due to the formation of oxidized lipids. Such conditions include, but are not limited to rheumatoid arthritis, lupus erythematous, polyarteritis nodosa, and osteoporosis.

In copending application PCT/US01/26497, filed on Aug. 23, 2001, we have shown that injecting apo A-I or one of the class A amphipathic helical peptide mimetics of apo A-I greatly ameliorated the signs and symptoms of influenza A in mice. The peptide mimetic described in this application dramatically reduced the influx of macrophages into the artery wall of the mice. This has great utility in reducing the high rate of heart attack and stroke after influenza and other viral infections.

We believe the phospholipids described herein act similarly. In particular, we believe that the synthetic phospholipids described herein also ameliorate the signs and symptoms of influenza and other viral illnesses and reduce the incidence of heart attack and stroke that often follows these viral illnesses.

A wide variety of lipids, preferably phospholipids are suitable for use in the methods of this invention. Such phospholipids can readily be determined by assaying a lipid candidate for its effect on lipoprotein cholesterol levels in apo E deficient mice (e.g., as described in Example 2), and/or on HDL protective capacity and LDL susceptibility to oxidation (e.g. as described in Example 2 and by Navab et al. (1997) J. Clin. Invest., 99: 2005-2019), and/or on fatty streak lesions ((e.g., as described in Example 2).

Particularly preferred lipids include phospholipids having fatty acids ranging from about 4 carbons to about 24 carbons in the sn-1 and sn-2 positions. In certain preferred embodiments, the fatty acids are saturated. In other preferred embodiments, the fatty acids can be unsaturated. In particularly preferred embodiments, the lipids are synthetic phospholipids such as synthetic phosphatidyl choline, or phosphatidyl serine, or phosphatidyl ethanolamine, or phosphatidyl inositol preferably containing one of the fatty acids listed below in Table 1 in the sn-1 position and another of the fatty acids listed below in Table 1 in the sn-2 position or one of these fatty acids in both the sn-1 and sn-2 positions. These synthetic phospholipids would be administered orally in doses averaging 0.1 g/day to 100 g/day, preferably 1 g/day to about 50 g/day, more preferably from about 2 g/day to about 20 or 10 g/day, and most preferably from about 3 or 6 g/day to about 12 to 20 g/day.

TABLE 1
Preferred fatty acids in the sn-1 or sn-2 positions of the preferred
phospholipids.

| Carbon No. | Common Name | IUPAC Name |
|---|---|---|
| 3:0 | Propionyl | Trianoic |
| 4:0 | Butanoyl | Tetranoic |
| 5:0 | Pentanoyl | Pentanoic |
| 6:0 | Caproyl | Hexanoic |
| 7:0 | Heptanoyl | Heptanoic |
| 8:0 | Capryloyl | Octanoic |
| 9:0 | Nonanoyl | Nonanoic |
| 10:0 | Capryl | Decanoic |
| 11:0 | Undecanoyl | Undecanoic |
| 12:0 | Lauroyl | Dodecanoic |
| 13:0 | Tridecanoyl | Tridecanoic |
| 14:0 | Myristoyl | Tetradecanoic |

We believe these lipids will be as safe or safer than existing practices and have the promise of being equally or more effective than existing practices and most important they may be additive to current practices.

VI. Mitigation of a Symptom of Atheroscloerosis Associated with an Acute Inflammatory Response.

The lipids described herein are also useful in a number of other contexts. In particular, we have observed that cardiovascular complications (e.g. atherosclerosis, stroke, etc.) frequently accompany or follow the onset of an acute phase inflammatory response. Such an acute state inflammatory response is often associated with a recurrent inflammatory disease (e.g., leprosy, tuberculosis, systemic lupus erythematosus, and rheumatoid arthritis), a viral infection (e.g. influenza), a bacterial infection, a fungal infection, an organ transplant, a wound or other trauma, an implanted prosthesis, a biofilm, and the like.

It was a surprising discovery of this invention that administration of one or more of the lipids described herein, can reduce or prevent the formation of oxidized phospholipids during or following an acute phase response and thereby mitigate or eliminate cardiovascular complications associated with such a condition.

Thus, for example, we have demonstrated that a consequence of influenza infection is the dimunition in paraoxonase and platelet activating acetylhydrolase activity in the HDL. Without being bound by a particular theory, we believe that, as a result of the loss of these HDL enzymatic activities and also as a result of the association of pro-oxidant proteins with HDL during the acute phase response, HDL is no longer able to prevent LDL oxidation and was no longer able to prevent the LDL-induced production of monocyte chemotactic activity by endothelial cells.

We observed that in a subject to whom one or more lipids are administered in accordance with this invention maintained active or elevated paraoxonase levels. This indicates that one or more lipids of this invention can be administered (e.g. orally or by injection) to patients with known coronary artery disease during influenza infection or other events that can generate an acute phase inflammatory response (e.g. due to viral infection, bacterial infection, trauma, transplant, various autoimmune conditions, etc.) and thus we can prevent by this short term treatment the increased incidence of heart attack and stroke associated with pathologies that generate such inflammatory states.

Thus, in certain embodiments, this invention contemplates administering one or more of the lipids of this invention to a subject at risk for, or incurring, an acute inflammatory response and/or at risk for or incurring a symptom of atherosclerosis.

Thus, for example, a person having or at risk for coronary disease may prophylactically be administered a lipid of this invention during flu season. A person (or animal) subject to a recurrent inflammatory condition, e.g. rheumatoid arthritis, various autoimmune diseases, etc., can be treated with a lipid of this invention to mitigate or prevent the development of atherosclerosis or stroke. A person (or animal) subject to trauma, e.g. acute injury, tissue transplant, etc. can be treated with a lipid of this invention to mitigate the development of atherosclerosis or stroke.

In certain instances such methods will entail a diagnosis of the occurrence or risk of an acute inflammatory response. The acute inflammatory response typically involves alterations in metabolism and gene regulation in the liver. It is a dynamic homeostatic process that involves all of the major systems of the body, in addition to the immune, cardiovascular and central nervous system. Normally, the acute phase response lasts only a few days; however, in cases of chronic or recurring inflammation, an aberrant continuation of some aspects of the acute phase response may contribute to the underlying tissue damage that accompanies the disease, and may also lead to further complications, for example cardiovascular diseases or protein deposition diseases such as amyloidosis.

An important aspect of the acute phase response is the radically altered biosynthetic profile of the liver. Under normal circumstances, the liver synthesizes a characteristic range of plasma proteins at steady state concentrations. Many of these proteins have important functions and higher plasma levels of these acute phase reactants (APRs) or acute phase proteins (APPs) are required during the acute phase response following an inflammatory stimulus. Although most APRs are synthesized by hepatocytes, some are produced by other cell types, including monocytes, endothelial cells, fibroblasts and adipocytes. Most APRs are induced between 50% and several-fold over normal levels. In contrast, the major APRs can increase to 1000-fold over normal levels. This group includes serum amyloid A (SAA) and either C-reactive protein (CRP) in humans or its homologue in mice, serum amyloid P component (SAP). So-called negative APRs are decreased in plasma concentration during the acute phase response to allow an increase in the capacity of the liver to synthesize the induced APRs.

In certain embodiments, the acute phase response, or risk therefore is evaluated by measuring one or more APPs. Measuring such markers is well known to those of skill in the art, and commercial companies exist that provide such measurement (e.g. AGP measured by Cardiotech Services, Louisville, Ky.).

VII. Kits.

In another embodiment, this invention provides kits for practicing one or more of the assays described herein or for therapeutic applications. Assay kits preferably comprise one or more containers containing a cell, tissue, or organ comprising a MKP-1 gene. The kits, optionally, include means for detecting expression of the MKP-1 gene product. Such means include, but are not limited to MKP-1 protein specific antibodies (labeled or unlabled), and/or MKP-1 gene or cDNA or mRNA specific probes (labeled or unlabeled), and/or one or more primers suitable for amplifying MKP-1 or a fragment thereof. Also optionally included is a mildly or highly oxidized LDL and/or a component thereof comprising an oxidized phospholipid, e.g. as described herein. The kits may optionally include devices and reagents to facilitate running the assays. Such devices and reagents include, but are not limited to microtiter plates (e.g. for high-throughput applications), buffers, labels, reagents for visualizing/detecting labels, software for running automated assays and/or acquiring and/or analyzing assay results, and the like.

Therapeutic kits preferably include one or more containers containing an agent capable of blocking and/or inhibiting MKP-1 expression and/or one of the lipids described herein. Agents may optionally include, but are not limited nucleic acids encoding antisense constructs, nucleic acids encoding intrabodies, nucleic acids encoding catalytic RNAs or catalytic DNAs, MKP-1 knockout constructs, small organic molecules that block or downregulate/inhibit MKP-1 expression/activity, inhibitory lipids, hydrophobic, or amphipathic molecules, and the like. The agents and/or lipids may be provided in a pharmaceutically acceptable excipient and or in a unit dose formulation.

In addition, the kits optionally include labeling and/or instructional materials providing directions (i.e., protocols) for the practice of the methods or use of the “therapeutics” of this invention. Preferred instructional materials describe screening for MKP-1 inhibitors as potential compounds for use in ameliorating one or more symptoms of atherosclerosis and/or rheumatoid arthritis. The methods may teach simple screens for MKP-1 inhibition and/or the use of oxidized LDLs or components thereof in the assays. In therapeutic/prophylactic kits, the instructional materials teach inhibition of MKP-1 as a treatment or prophylactic to ameliorate one or more symptoms of atherosclerosis, and/or associated pathologies and/or rheumatoid arthritis. Certain instructional materials may teach the use/administration of lipids to mitigate one or more symptoms of atherosclerosis and/or to mitigate one or more symptoms of another pathology characterized by an inflammatory response. The instructional materials may also, optionally, teach preferred dosages/therapeutic regiment, counterindications and the like.

While the instructional materials typically comprise written or printed materials they are not limited to such. Any medium capable of storing such instructions and communicating them to an end user is contemplated by this invention. Such media include, but are not limited to electronic storage media (e.g., magnetic discs, tapes, cartridges, chips), optical media (e.g., CD ROM), and the like. Such media may include addresses to internet sites that provide such instructional materials.

### EXAMPLES

The following examples are offered to illustrate, but not to limit the claimed invention.

### Example 1

### MKP-1 is Upregulated in Response to Mildly or Highly Oxidized LDL or Components Thereof

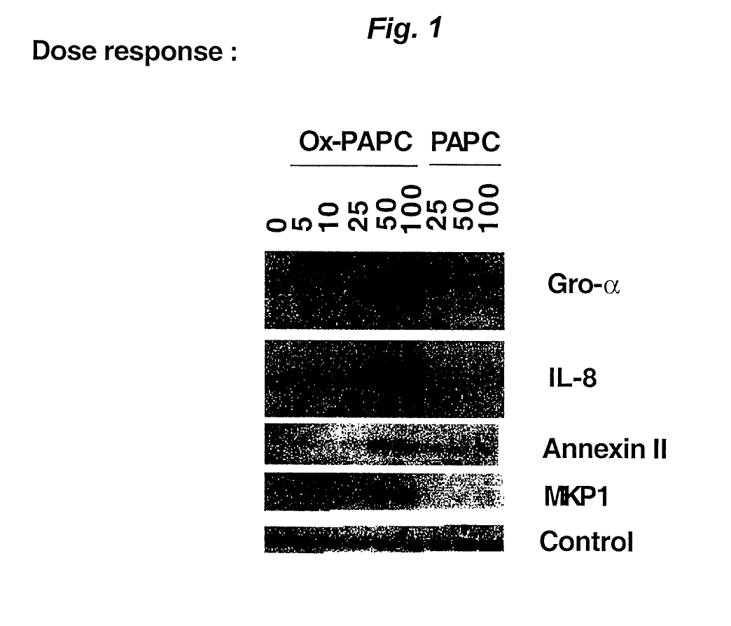

Confluent cultures of human aortic endothelial cells (HAECs) were treated for 4 hours with various concentrations (as shown in FIG. 1) of Ox-PAPC or PAPC. Following the treatments, cells were lysed and total RNA was isolated. 10 μg of total RNA from each condition was subjected gel electrophoresis and transferred to a nitrocellulose membrane. The immobilized RNA was hybridized to radiolabeled cDNA for human Gro-α, IL-8, Annexin II, MKP-1 and a control gene (GAPDH).

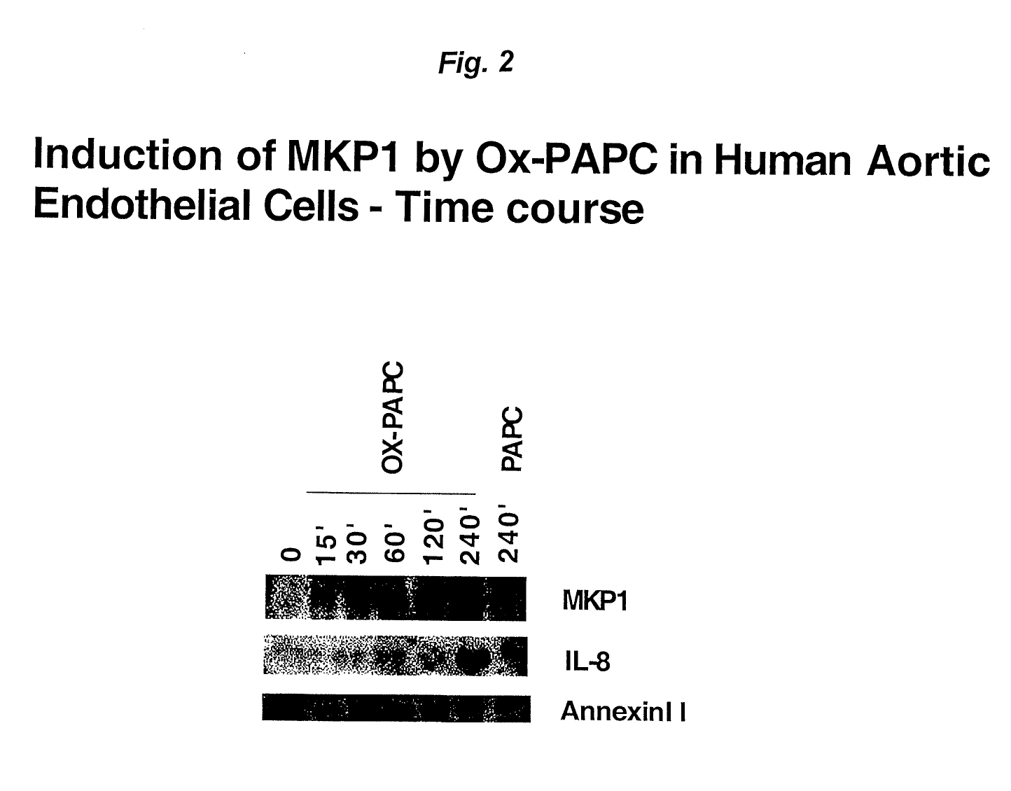

In another experiment, confluent cultures of HAECs were treated with Ox-PAPC (50 μg/ml) or PAPC (50 μg/ml). At various time points (as indicated in FIG. 2) cells were lysed and total RNA was isolated. 10 μg of total RNA from each condition was subjected gel electrophoresis and transferred to a nitrocellulose membrane. The immobilized RNA was hybridized to radiolabeled cDNA for human IL-8, Annexin II and MKP-1.

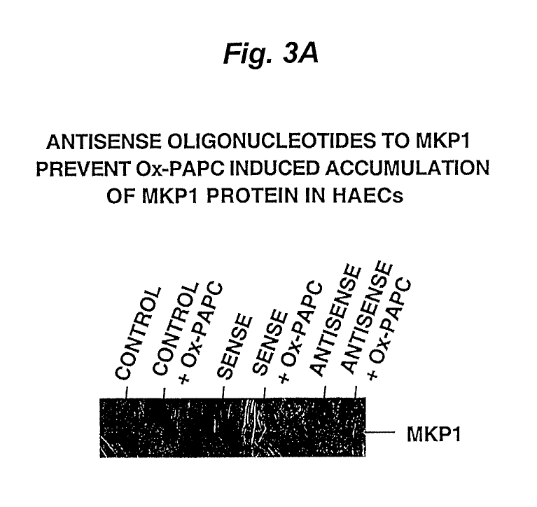

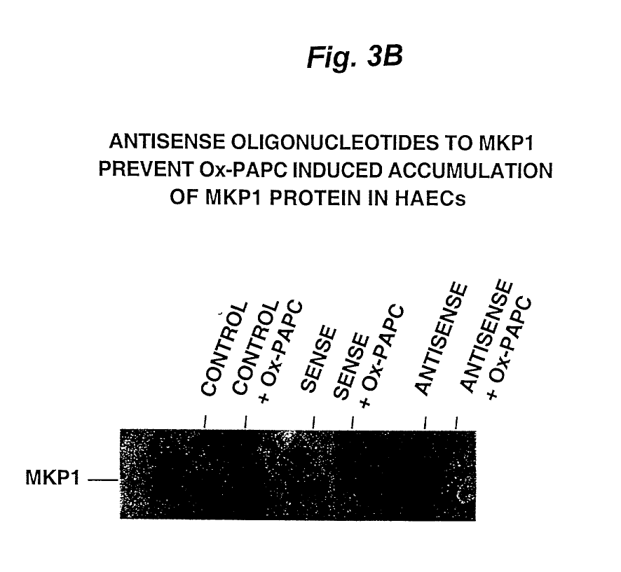

HAECs were transfected with either ‘antisense’ or ‘sense’ phosphorothioate oligonucleotides (100 nM) to human MKP-1. Eighteen hours later, control and transfected cells were either left untreated or treated with Ox-PAPC (50 μg/ml) for an additional four hours. Following the treatments, cell lysates were prepared and analyzed by western blotting for MKP-1 protein expression As shown in figure FIG. 3A and FIG. 3A 1 antisense oligonucleotides prevent the accumulation of MKP-1 protein in Ox-PAPC induced HAECs.

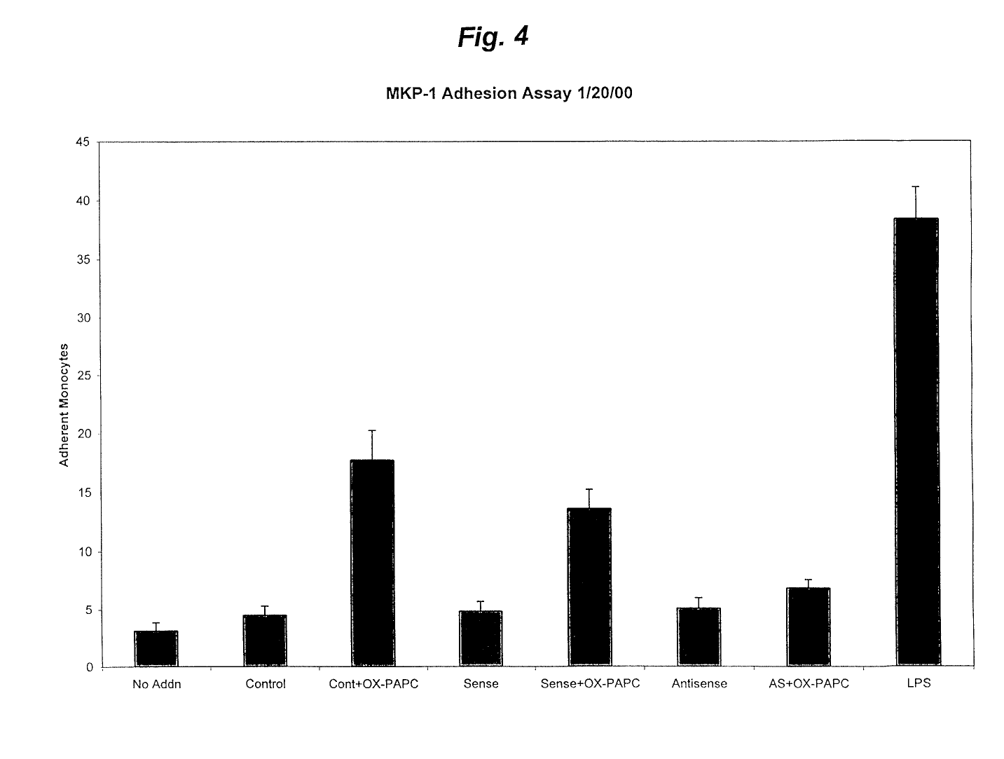

Human aortic endothelial cells were transfected with either ‘antisense’ or ‘sense’ phosphorothioate oligonucleotides (100 nM) to human MKP-1. Eighteen hours later, control and transfected cells were either left untreated or treated with Ox-PAPC (50 μg/ml) for an additional four hours. Following the treatments, cell supernatants were collected and analyzed in a monocyte adhesion assay. As illustrated in FIG. 4 supernatants from Ox-PAPC treated HAECs pretreated with antisense oligonucleotides to MKP-1 did not promote monocyte adhesion.

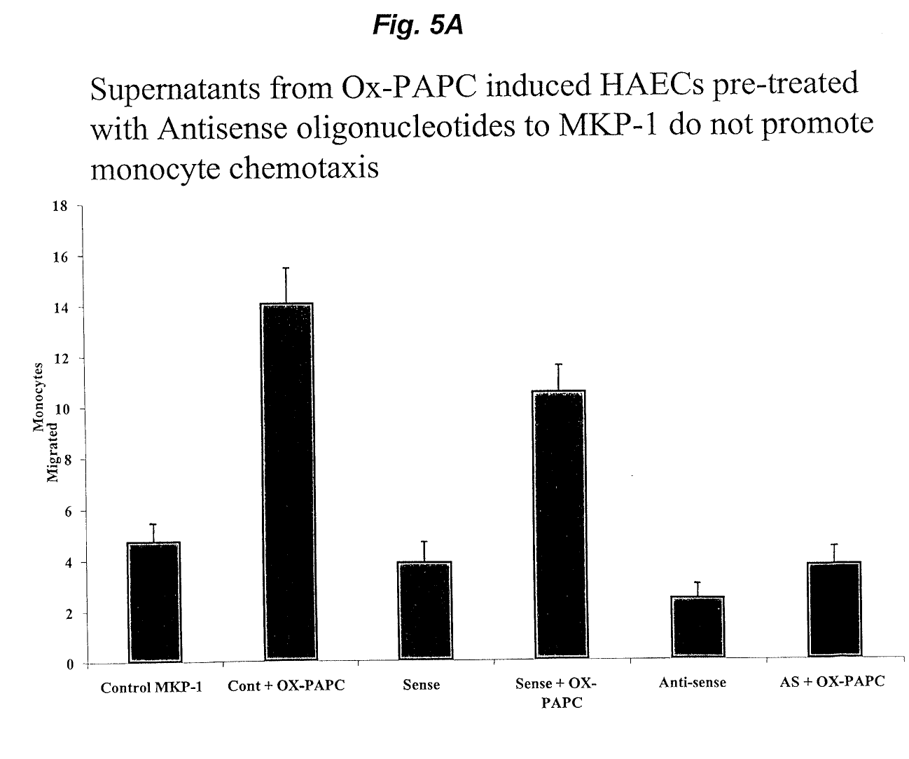

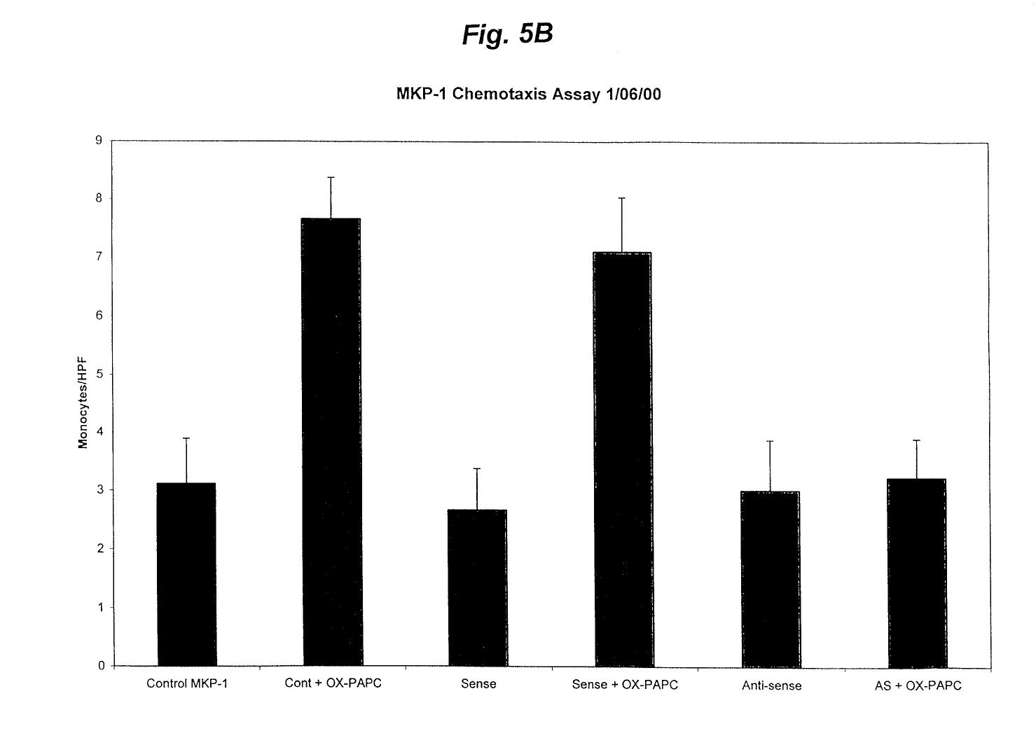

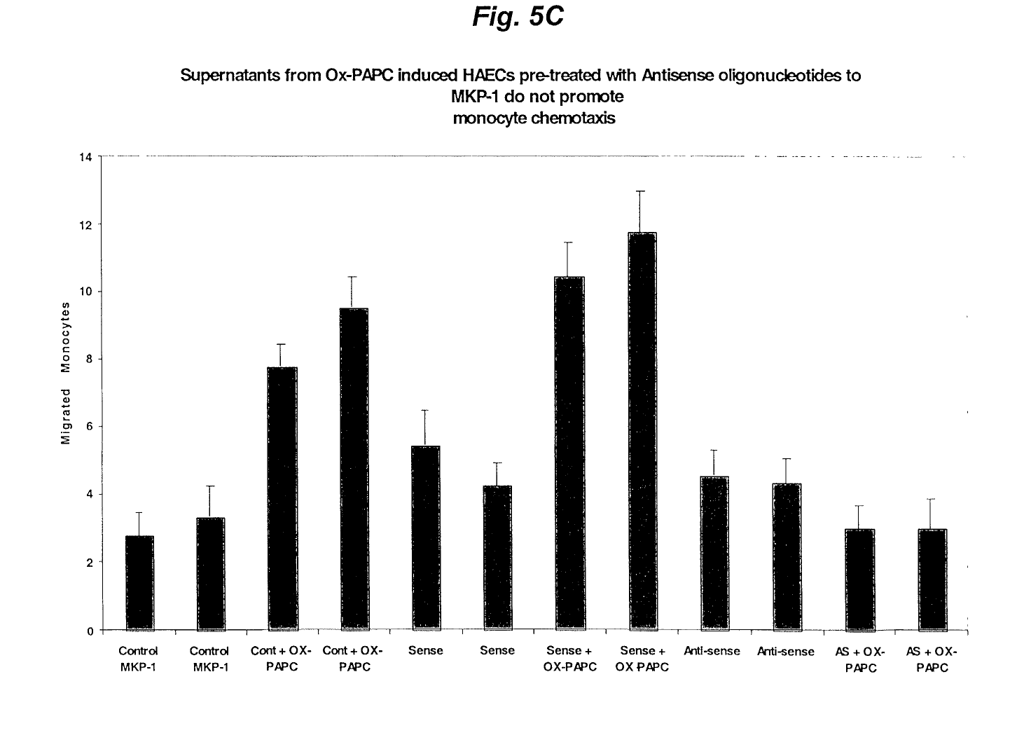

Human aortic endothelial cells were transfected with either ‘antisense’ or ‘sense’ phosphorothioate oligonucleotides (100 nM) to human MKP-1. Eighteen hours later, control and transfected cells were either left untreated or treated with Ox-PAPC (50 μg/ml) for an additional four hours. Following the treatments, cell supernatants were collected and analyzed for monocyte chemotactic activity. As shown in FIG. 5A, FIG. 5B, and FIG. 5C, supernatants from Ox-PAPC induced HAECs pretreated with antisense oligonucleotides to MKP-1 did not promote monocyte chemotaxis.

### Example 2

### Ameliorate Atherosclerosis and Other Inflammatory Conditions

FIG. 6 shows the effect of 1,2-dimyristoyl-sn-glycero-3-phosphocholine (1,2-ditetradecanoyl-sn-glycero-3-phosphocholine) and egg yolk lecithin on lipoprotein cholesterol levels in apo E deficient mice. Apo E deficient mice were purchased from Jackson Laboratories (Bar Harbor, Me.). The mice were 5 weeks old at the time of the experiments. The animals were maintained on a chow diet containing 4% fat, obtained from Ralston-Purina (St. Louis, Mo.). Synthetic 1,2-ditetradecanoyl-sn-glycero-3-phosphocholine (shown in the figure as circles at 24 h and as triangles at 48 h and abbreviated as DGP; 99+% purity, catalog # P 2663, Lot # 17H8362, Sigma Chemical Company, St. Louis Mo.) was added at 2.0 mg/ml to the drinking water of one group of apo E deficient mice (n=5). The second group (n=5) were given 2.0 mg/ml of egg yolk lecithin (Sigma catalog # P 7318) in their drinking water (shown as squares and only the 48 h time point is displayed). The control group (n=5) did not receive any supplement in the drinking water (shown as diamonds and only the 48 h time point is displayed). After 24 and 48 hrs blood was collected from the retroorbital sinus under mild anesthesia into heparinized tubes under an approved protocol of the UCLA Animal Research Committee. Plasma was separated and was fractionated using fast protein liquid chromatography (FPLC). Cholesterol content of the fractions was determined using a kit (Infinity reagent) obtained from Sigma Chemical Company.

FIG. 7 shows the effect of 1,2-dimyristoyl-sn-glycero-3-phosphocholine (1,2-ditetradecanoyl-sn-glycero-3-phosphocholine) (shown in the figure as squares and abbreviated as DGP) and egg yolk lecithin (shown in the figure as asterisks) on paraoxonase activity. Plasma fractions obtained as described in above were analyzed for paraoxonase activity using paraoxon a substrate as reported by Watson et al. (1995) J Clin Invest., 96: 2882-2891). The control samples are shown as diamonds in the figure.

When 1,2-dimyristoyl-sn-glycero-3-phosphocholine (1,2-ditetradecanoyl-sn-glycero-3-phosphocholine) was added to the drinking water of mice that have been genetically engineered to spontaneously develop atherosclerosis on a low fat diet (Apo E null mice) there was a dramatic increase in the high density lipoprotein (HDL) levels in the mice without a significant change in the atherogenic lipoproteins very low density lipoproteins (VLDL) or low density lipoproteins (LDL) (see FIG. 6). There was also a dramatic increase in the content of an atheroprotecitive protein associated with HDL, paraoxonase-1 (PON1) (see FIG. 7).

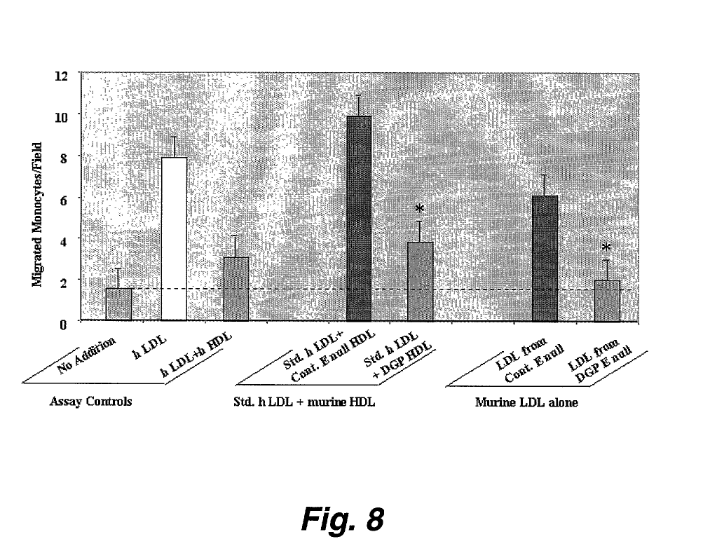

FIG. 8 shows the effect of 1,2-dimyristoyl-sn-glycero-3-phosphocholine (1,2-ditetradecanoyl-sn-glycero-3-phosphocholine) on HDL protective capacity and LDL susceptibility to oxidation. Plasma fractions obtained as described above were pooled in the region containing LDL and the region containing HDL. Human artery wall cell cocultures were prepared as reported previously (Navab et al. (1997) J Clin Invest., 99: 2005-2019) and used to determine the extent of LDL oxidation as judged by the level of monocyte chemotactic activity generated by oxidized LDL. As the assay controls, to some wells containing the artery wall cocultures was added a standard human LDL (h LDL) at 200 μg LDL protein per ml. To other wells was added h LDL +human HDL (h HDL) at 350 μg HDL protein per ml. The protective capacity of HDL obtained from apo E deficient mice was then tested. To some wells was added standard human LDL (Std. h LDL) at 200 μg LDL protein per ml plus 50 μg of HDL cholesterol obtained from the mice that had received drinking water without additions (Std. h LDL +Cont. E null HDL). To other wells in addition to human LDL, HDL (at 50 μg cholesterol/ml) obtained from mice given drinking water containing 2.0 mg per ml of 1,2-dimyristoyl-sn-glycero-3-phosphocholine (1,2-ditetradecanoyl-sn-glycero-3-phosphocholine) (Std. h LDL +DGP HDL) was added and incubated for 18 hrs. LDL susceptibility to oxidation was additionally determined. LDL obtained from the apo E deficient mice maintained on the drinking water without phospholipid supplement (LDL from Cont. E null) or LDL from the mice given drinking water containing 2.0 mg per ml of 1,2-dimyristoyl-sn-glycero-3-phosphocholine (1,2-ditetradecanoyl-sn-glycero-3-phosphocholine) (LDL from DGP E null) were added at 100 μg LDL cholesterol per ml and incubated for 18 hrs. The supernatants were collected, diluted 40 fold and assayed for monocyte chemotactic activity as described in the reference stated above. The values are mean ±SD of the number of monocytes counted in 9 high power fields from quadruple wells. Asterisks indicate significant differences as compared with controls at a level of p<0.001.

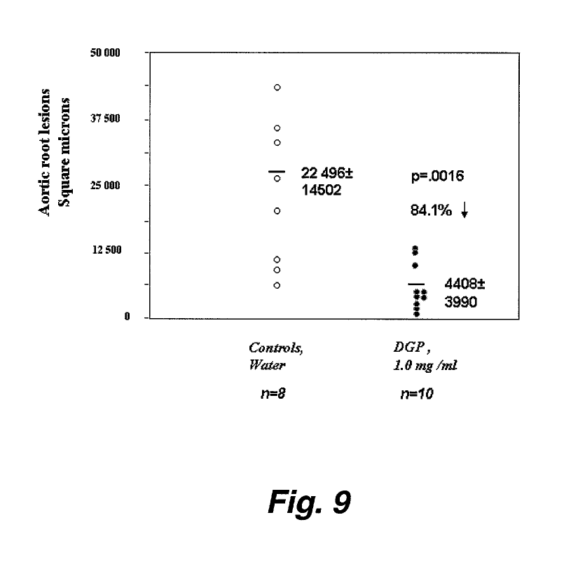

FIG. 9 shows the effect of 1,2-dimyristoyl-sn-glycero-3-phosphocholine (1,2-ditetradecanoyl-sn-glycero-3-phosphocholine) on fatty streak lesions. Apo E deficient mice were purchased from Jackson Laboratories (Bar Harbor, Me.). The mice were 5 weeks old at the time of the experiments. The animals were maintained on a chow diet containing 4% fat, obtained from Ralston-Purina (St. Louis, Mo.). One group (n=10) was given in their drinking water 1.0 mg/ml of 1,2-dimyristoyl-sn-glycero-3-phosphocholine (1,2-ditetradecanoyl-sn-glycero-3-phosphocholine) (shown as DGP on the figure, approximately 99% pure, catalog # P 7331, Sigma Chemical Company, St. Louis, Mo.). The control group (n=8) did not receive any supplements in the drinking water. After 5 weeks of treatment, the animals were sacrificed and hearts were harvested and processed according to the methods described by Qiao et al. (1994) Arterioscl Thromb, 14: 1480-1497). In brief, 10 μm thick cryosections were stained with Oil red 0 and hematoxylin and counterstained with fast green and examined by light microscopy for the identification of atheromatous lesions. Lesion size was measured using a 1.0 cm optical grid with 20×20 grid lines. Under 10× magnification, each square corresponded to 1/400 mm² (or 2500 μm²). A square with lipophilic stain was counted as lesion if the visually observed stain covered at least ⅛th of the square. The measure of lesion size was derived from the number of squares counted over the 20 consecutive stained sections superior to the appearance of the aortic valve.

The mice that received the lipid in their drinking water had a 84.1% reduction in atherosclerosis

In summary, 1,2-dimyristoyl-sn-glycero-3-phosphocholine (1,2-ditetradecanoyl-sn-glycero-3-phosphocholine) by itself was able to raise HDL levels, paraoxonase activity (a protective enzyme associated with HDL), was able to render HDL more effective in protecting LDL against oxidation and rendered LDL more resistant to oxidation by artery walls and dramatically reduced atherosclerosis in genetically engineered mice that develop atherosclerosis resembling human atherosclerosis while the mice are eating a low fat chow diet.

### Example 3

### Ditetradecanoyl Glycero Phosphorylcholine Causes Lesion Regression

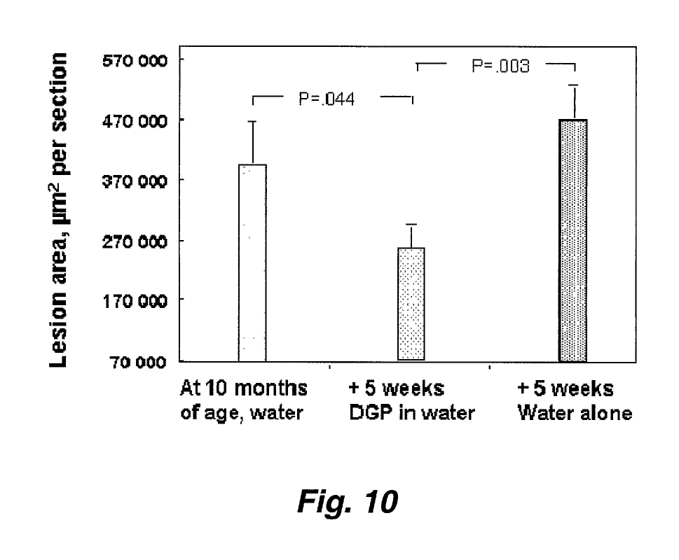

Apo E deficient mice were purchased from Jackson Laboratories (Bar Harbor, Me.). The mice were maintained on a chow diet containing 4% fat, obtained from Ralston-Purina (St. Louis, Mo.) for 10 months at which time one group (n=5) were sacrificed and lesions determined. A second group (n=8) received 1.0 mg/ml of synthetic 1,2-ditetradecanoyl-sn-glycero-3-phosphorylcholine (DGP, approximately 99% pure, catalog # P 7331, Sigma Chemical Company, St. Louis, Mo.) in their drinking water for another five weeks. A control group (n=6) did not receive any supplements in their drinking water. After 5 weeks of treatment, the animals were sacrificed. The hearts and blood vessels from the three groups were harvested and processed as previously reported (Qiao 1994) to determine lesion area. As expected the mice that received drinking water alone showed progression of their lesions during the additional five weeks (see FIG. 10). In contrast, the mice that received DGP in their drinking water showed dramatic lesion regression (see FIG. 10).

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

What is claimed is:

1. A method of ameliorating one or more symptoms of atherosclerosis in a mammal, said method comprising administering to said mammal a phospholipid in an amount sufficient to ameliorate one or more symptoms of atherosclerosis.

2. The method of claim 1, wherein said phospholipid is a phospholipid that inhibits upregulation of an MKP-1 gene.

3. The method of claim 1, wherein said phospholipid is a phospholipid selected from the group consisting of phosphatidyl choline, phosphatidyl serine, phosphatidyl ethanolamine, and phosphatidyl inositol, and said phospholipid comprises independently selected fatty acids in the sn-1 and sn-2 positions ranging in length from about 4 to about 24 carbons.

4. The method of claim 3, wherein the fatty acids in in the sn-1 and sn-2 positions are independently selected from the group consisting of propionoyl, butanoyl, pentanoyl, caproyl, heptanoyl, capryloyl, nonanoyl, capryl, undcanoyl, lauroyl, tridecanoyl, and myristoyl.

5. The method of claim 1, wherein said phospholipid is 1,2-dimyristoyl-sn-glycero-3-phosphocholine or an analogue thereof.

6. The method of claim 1, wherein said phospholipid is provided in a unit dose form.

7. The method of claim 1, wherein said phospholipid is provided in a combination of phospholipids.

8. The method of claim 1, where said administration is by a route selected from the group consisting of oral administration, nasal administration, rectal administration, intraperitoneal injection, and intravascular injection, subcutaneous injection, transcutaneous administration, and intramuscular injection.

9. The method of claim 1, wherein said administration is oral administration.

10. The method of claim 1, wherein said administration is an injection.

11. The method of claim 1, wherein said symptoms are in a human patient diagnosed as having or at risk for atherosclerosis.

12. The method of claim 1, wherein said symptoms are in a human patient diagnosed as having atherosclerosis.

13. The method of claim 1, wherein said mammal is also administered a statin.

14. The method of claim 1, wherein said mammal is not administered a synthetic peptide.

15. The method of claim 1, wherein said phospholipid is administered in a formulation lacking other active agents.

16. A method of mitigating or preventing a coronary complication associated with an acute phase response to an inflammation in a mammal, wherein said coronary complication is a symptom of atherosclerosis, said method comprising administering to a mammal having said acute phase response, or at risk for said acute phase response, a phospholipid in an amount sufficient to mitigate or prevent said coronary complication.

17. The method of claim 16, wherein said phospholipid is a phospholipid selected from the group consisting of phosphatidyl choline, phosphatidyl serine, phosphatidyl ethanolamine, and phosphatidyl inositol, and said phospholipid comprises independently selected fatty acids in the sn-1 and sn-2 positions ranging in length from about 4 to about 24 carbons.

18. The method of claim 17, wherein the fatty acids in in the sn-1 and sn-2 positions are independently selected from the group consisting of propionoyl, butanoyl, pentanoyl, caproyl, heptanoyl, capryloyl, nonanoyl, capryl, undcanoyl, lauroyl, tridecanoyl, and myristoyl.

19. The method of claim 16, wherein said phospholipid is 1,2-dimyristoyl-sn-glycero-3-phosphocholine or an analogue thereof.

20. The method of claim 16, wherein said phospholipid is provided in a unit dose form.

21. The method of claim 16, wherein said phospholipid is provided in a combination of phospholipids.

22. The method of claim 16, where said administration is by a route selected from the group consisting of oral administration, nasal administration, rectal administration, intraperitoneal injection, and intravascular injection, subcutaneous injection, transcutaneous administration, and intramuscular injection.

23. The method of claim 16, wherein said administration is oral administration.

24. The method of claim 16, wherein said administration is an injection.

25. The method of claim 16, wherein said acute phase response is an inflammatory response associated with a recurrent inflammatory disease.

26. The method of claim 16, wherein said acute phase response is associated with a disease selected from the group consisting of leprosy, tuberculosis, systemic lupus erythematosus, polymyalgia rheumatica, polyarteritis nodosa, scleroderma, idiopathic pulmonary fibrosis, chronic obstructive pulmonary disease, Alzheimers Disease AIDS, coronary calcification, calcific aortic stenosis, osteoporosis, and rheumatoid arthritis.

27. The method of claim 16, wherein said acute phase response is an inflammatory response associated with a condition selected from the group consisting of a bacterial infection, a viral infection, a fungal infection, an organ transplant, a wound, an implanted prosthesis, parasitic infection, sepsis, endotoxic shock syndrome, and biofilm formation.

28. A method of mitigating or preventing a coronary complication associated with an acute phase response to an inflammation in a mammal, wherein said coronary complication is a symptom of atherosclerosis, said method comprising:
assaying said mammal for an acute phase protein (APP) level indicative of an acute phase response or a significant risk of an acute phase response; and
administering to a mammal showing an acute phase protein (APP) level indicative of an acute phase response a phospholipid in an amount sufficient to mitigate or prevent said coronary complication.

29. The method of claim 28, wherein said phospholipid is a phospholipid selected from the group consisting of phosphatidyl choline, phosphatidyl serine, phosphatidyl ethanolamine, and phosphatidyl inositol, and said phospholipid comprises independently selected fatty acids in the sn-1 and sn-2 positions ranging in length from about 4 to about 24 carbons.

30. The method of claim 29, wherein the fatty acids in in the sn-1 and sn-2 positions are independently selected from the group consisting of propionoyl, butanoyl, pentanoyl, caproyl, heptanoyl, capryloyl, nonanoyl, capryl, undcanoyl, lauroyl, tridecanoyl, and myristoyl.

31. The method of claim 29, wherein said phospholipid is 1,2-dimyristoyl-sn-glycero-3-phosphocholine or an analogue thereof.

32. The method of claim 28, wherein said acute phase response is an inflammatory response associated with a recurrent inflammatory disease.

33. The method of claim 28, wherein said acute phase response is associated with a disease selected from the group consisting of leprosy, tuberculosis, systemic lupus erythematosus, polymyalgia rheumatica, polyarteritis nodosa, scleroderma, idiopathic pulmonary fibrosis, chronic obstructive pulmonary disease, Alzheimers Disease AIDS, coronary calcification, calcific aortic stenosis, osteoporosis, and rheumatoid arthritis.

34. The method of claim 28, wherein said acute phase response is an inflammatory response associated with a condition selected from the group consisting of a bacterial infection, a viral infection, a fungal infection, an organ transplant, a wound, an implanted prosthesis, parasitic infection, sepsis, endotoxic shock syndrome, and biofilm formation.

35. The method of claim 28, wherein said acute phase protein (APP) is a positive APR selected from the group consisting of serum amyloid A, c-reactive protein, serum amyloid P component, C2 complement protein, C3 complement protein, C4 complement protein, C5 complement protein, C9 complement protein, B complement protein, C1 inhibitor, C4 binding protein, fibrinogen, von Willebrand factor, α1-antitrypsin, α1-antichymotrypsin, α2 antiplasmin, heparin cofactor II, plasminogen activator inhibitor I, haptoglobin, haemopexin, ceruloplasmin, manganese superoxide dismutase, α1-acid glycoprotein, haeme oxygenase, mannose binding protein, leukocyte protein I, lipoprotein (a), and lipopolysaccharide binding protein.

36. The method of claim 28, wherein said acute phase protein (APP) is a negative APR selected from the group consisting of albumin, prealbumin, transferin, apoAI, apoAII, α2-HS glycoprotein, inter-α-trypsin inhibitor, histidine rich glycoprotein.

37. The method of claim 28, wherein said phospholipid is provided in a unit dose form.

38. The method of claim 28, wherein said phospholipid is provided in a combination of phospholipids.

39. The method of claim 28, where said administration is by a route selected from the group consisting of oral administration, nasal administration, rectal administration, intraperitoneal injection, and intravascular injection, subcutaneous injection, transcutaneous administration, and intramuscular injection.

40. The method of claim 28, wherein said administration is oral administration.

41. The method of claim 28, wherein said administration is an injection.

42. A method of inhibiting a symptom of an inflammatory condition, said method comprising administering to a mammal exhibiting a symptom of a pathology characterized by an inflammatory response a phospholipid in an amount sufficient to mitigate a symptom associated with said inflammatory condition.

43. The method of claim 42, wherein said phospholipid is a phospholipid selected from the group consisting of phosphatidyl choline, phosphatidyl serine, phosphatidyl ethanolamine, and phosphatidyl inositol, and said phospholipid comprises independently selected fatty acids in the sn-1 and sn-2 positions ranging in length from about 4 to about 24 carbons.

44. The method of claim 43, wherein the fatty acids in the sn-1 and sn-2 positions are independently selected from the group consisting of propionoyl, butanoyl, pentanoyl, caproyl, heptanoyl, capryloyl, nonanoyl, capryl, undcanoyl, lauroyl, tridecanoyl, and myristoyl.

45. The method of claim 42, wherein said phospholipid is 1,2-dimyristoyl-sn-glycero-3-phosphocholine or an analogue thereof.

46. The method of claim 42, wherein said phospholipid is provided in a unit dose form.

47. The method of claim 42, wherein said phospholipid is provided in a combination of phospholipids.

48. The method of claim 42, where said administration is by a route selected from the group consisting of oral administration, nasal administration, rectal administration, intraperitoneal injection, and intravascular injection, subcutaneous injection, transcutaneous administration, and intramuscular injection.

49. The method of claim 42, wherein said symptoms are in a human patient diagnosed as having or at risk for atherosclerosis.

50. The method of claim 42, wherein said symptoms are in a human patient diagnosed as having atherosclerosis.

51. The method of claim 52, wherein said inflammatory condition is selected from the group consisting of rheumatoid arthritis, lupus erythematous, polyarteritis nodosa, osteoporosis, and a viral illness.

52. A composition for ameliorating one or more symptoms of atherosclerosis, said composition comprising a phospholipid in a unit dose form.

53. The composition of claim 52, wherein said unit dose form further comprises a pharmacologically acceptable excipient.

54. The composition of claim 52, wherein said phospholipid is a phospholipid selected from the group consisting of phosphatidyl choline, phosphatidyl serine, phosphatidyl ethanolamine, and phosphatidyl inositol, and said phospholipid comprises independently selected fatty acids in the sn-1 and sn-2 positions ranging in length from about 4 to about 24 carbons.

55. The composition of claim 54, wherein the fatty acids in the sn-1 and sn-2 positions are independently selected from the group consisting of propionoyl, butanoyl, pentanoyl, caproyl, heptanoyl, capryloyl, nonanoyl, capryl, undcanoyl, lauroyl, tridecanoyl, and myristoyl.

56. The composition of claim 52, wherein said phospholipid is 1,2-dimyristoyl-sn-glycero-3-phosphocholine or an analogue thereof.

57. A kit for ameliorating one or more symptoms of atherosclerosis, said kit comprising:
a phospholipid; and
instructional materials teaching the administration of said phospholipid to a mammal to mitigate one or more symptoms of atherosclerosis.

58. The kit of claim 57, wherein said phospholipid is a phospholipid selected from the group consisting of phosphatidyl choline, phosphatidyl serine, phosphatidyl ethanolamine, and phosphatidyl inositol, and said phospholipid comprises independently selected fatty acids in the sn-1 and sn-2 positions ranging in length from about 4 to about 24 carbons.

59. The kit of claim 58, wherein the fatty acids in the sn-1 and sn-2 positions are independently selected from the group consisting of propionoyl, butanoyl, pentanoyl, caproyl, heptanoyl, capryloyl, nonanoyl, capryl, undcanoyl, lauroyl, tridecanoyl, and myristoyl.

60. The kit of claim 57, wherein said phospholipid is 1,2-dimyristoyl-sn-glycero-3-phosphocholine or an analogue thereof.

61. The kit of claim 57, wherein said phospholipid is in a unit dose form.

62. A kit for inhibiting a symptom of an inflammatory condition, said kit comprising:
a phospholipid; and
instructional materials teaching the administration of said phospholipid to a mammal to mitigate one or more symptoms of an inflammatory condition.

63. The kit of claim 66, wherein said phospholipid is a phospholipid selected from the group consisting of phosphatidyl choline, phosphatidyl serine, phosphatidyl ethanolamine, and phosphatidyl inositol, and said phospholipid comprises independently selected fatty acids in the sn-1 and sn-2 positions ranging in length from about 4 to about 24 carbons.

64. The kit of claim 63, wherein the fatty acids in the sn-1 and sn-2 positions are independently selected from the group consisting of propionoyl, butanoyl, pentanoyl, caproyl, heptanoyl, capryloyl, nonanoyl, capryl, undcanoyl, lauroyl, tridecanoyl, and myristoyl.

65. The kit of claim 66, wherein said phospholipid is 1,2-dimyristoyl-sn-glycero-3-phosphocholine or an analogue thereof.

66. The kit of claim 66, wherein said phospholipid is in a unit dose form.

67. A method of repairing tissue damage associated with atherosclerosis, said method comprising administering to said mammal a phospholipid in an amount sufficient to partially or fully repair tissue damage to a tissue wherein said damage is tissue damage associated with atherosclerosis.

68. The method of claim 67, wherein said phospholipid is a phospholipid that inhibits upregulation of an MKP-1 gene.

69. The method of claim 67, wherein said phospholipid is a phospholipid selected from the group consisting of phosphatidyl choline, phosphatidyl serine, phosphatidyl ethanolamine, and phosphatidyl inositol, and said phospholipid comprises independently selected fatty acids in the sn-1 and sn-2 positions ranging in length from about 4 to about 24 carbons.

70. The method of claim 69, wherein the fatty acids in in the sn-1 and sn-2 positions are independently selected from the group consisting of propionoyl, butanoyl, pentanoyl, caproyl, heptanoyl, capryloyl, nonanoyl, capryl, undcanoyl, lauroyl, tridecanoyl, and myristoyl.

71. The method of claim 67, wherein said phospholipid is 1,2-dimyristoyl-sn-glycero-3-phosphocholine or an analogue thereof.

72. The method of claim 67, wherein said phospholipid is provided in a unit dose form.

73. The method of claim 67, wherein said phospholipid is provided in a combination of phospholipids.

74. The method of claim 67, where said administration is by a route selected from the group consisting of oral administration, nasal administration, rectal administration, intraperitoneal injection, and intravascular injection, subcutaneous injection, transcutaneous administration, and intramuscular injection.

75. The method of claim 67, wherein said administration is oral administration.

76. The method of claim 67, wherein said administration is an injection.

77. The method of claim 67, wherein said symptoms are in a human patient diagnosed as having or at risk for atherosclerosis.

78. The method of claim 67, wherein said symptoms are in a human patient diagnosed as having atherosclerosis.

79. The method of claim 67, wherein said mammal is also administered a statin.

80. The method of claim 67, wherein said mammal is not administered a synthetic peptide.

81. The method of claim 67, wherein said phospholipids is administred in a formulation lacking other active agents.
